SEQUENCE LISTING (2 sequences)

SEQ ID NO: 1 - DNA, 21 bp, Artificial Sequence
  Description of Artificial Sequence Synthetic oligonucleotide
cttacgctgagtacttcgatt

SEQ ID NO: 2 - DNA, 21 bp, Artificial Sequence
  Description of Artificial Sequence Synthetic oligonucleotide
tcgaagtactcagcgtaagtt

(19) United States
(12) Patent Application Publication
(10) Pub. No.: US 2011/0288152 A1
(43) Pub. Date: Nov. 24, 2011

(54) PSMA BINDING LIGAND-LINKER CONJUGATES AND METHODS FOR USING

(75) Inventors: Philip Stewart Low, West Lafayette, IN (US); Sumith A. Kularatne, San Diego, CA (US)

(73) Assignee: Purdue Research Foundation, West Lafayette, IN (US)

(21) Appl. No.: 13/124,448

(22) PCT Filed: Oct. 16, 2009
(86) PCT No.: PCT/US09/61067

§ 371 Date: Apr. 15, 2011

Related U.S. Application Data
(60) Provisional application No. 61/196,488, filed on Oct. 17, 2008.

(51) Int. Cl. A61K 31/7088; A61P 35/00; C07K 2/00; A61K 31/713; C07H 21/04; C07H 21/02

(52) U.S. Cl. 514/44 A; 536/23.1; 536/24.5; 530/322; 514/44 R

(57) ABSTRACT

Described herein are prostate specific membrane antigen (PSMA) binding conjugates that are useful for targeting prostate cancer cells. Also described herein are compositions containing them and methods of using the conjugates and compositions. Also described are processes for manufacture of the conjugates and the compositions containing them.

### CROSS REFERENCE TO RELATED APPLICATIONS

This application claims priority under 35 USC §119(e) to U.S. Provisional Application Ser. No. 61/196,488, filed on Oct. 17, 2008, the entire disclosure of each of which is incorporated herein by reference.

### TECHNICAL FIELD

The invention described herein pertains to compounds and methods for targeting nucleotides to prostate cancer cells. More specifically, embodiments of the invention described herein pertain to conjugates of nucleotides conjugated to PSMA binding ligands for use in specific targeting of the conjugate to prostate cancer cells.

### BACKGROUND

The mammalian immune system provides a means for the recognition and elimination of tumor cells, other pathogenic cells, and invading foreign pathogens. While the immune system normally provides a strong line of defense, there are many instances where cancer cells or other pathogenic cells evade a host immune response and proliferate or persist with concomitant host pathogenicity. Chemotherapeutic agents and radiation therapies have been developed to eliminate, for example, replicating neoplasms. However, many of the currently available chemotherapeutic agents and radiation therapy regimens have adverse side effects because they work not only to destroy pathogenic cells, but they also affect normal host cells, such as cells of the hematopoietic system.

Researchers have developed therapeutic protocols for destroying pathogenic cells by targeting cytotoxic compounds to such cells. Many of these protocols utilize toxins conjugated to antibodies that bind to antigens unique to or overexpressed by the pathogenic cells in an attempt to minimize delivery of the toxin to normal cells. Using this approach, certain immunotoxins have been developed consisting of antibodies directed to specific antigens on pathogenic cells, the antibodies being linked to toxins such as ricin, Pseudomonas exotoxin, Diptheria toxin, and tumor necrosis factor. These immunotoxins target pathogenic cells, such as tumor cells, bearing the specific antigens recognized by the antibody (Olsnes, S., Immunol. Today, 10, pp. 291-295, 1989; Melby, E. L., Cancer Res., 53(8), pp. 1755-1760, 1993; Better, M. D., PCT Publication Number WO 91/07418, published May 30, 1991). However, antibody conjugates are expensive to produce, and their large size and affinity for serum proteins may result in reduced delivery to the tumor. The side effects of chemotherapeutic agents and radiation, and the disadvantages of antibody conjugates highlight the need for the development of new conjugates selective for pathogenic cell populations and with reduced host toxicity.

Small interfering RNA (siRNA) is a class of short (e.g., 20 to 30 nucleotides), double stranded RNA molecules that play a variety of roles in the regulation of genes and corresponding proteins. siRNAs are well-defined double stranded RNA structures with 2-nucleotide 3′ overhangs on either end. Each siRNA strand has a 5′ phosphate group and a 3′ hydroxyl group. This structure is the result of processing by dicer, an enzyme that converts either long dsRNAs or small hairpin RNAs into siRNAs. siRNAs can also be exogenously introduced into cells by various methods to bring about the specific knockdown of a gene of interest. For example, any gene of which the sequence in known can be targeted based on sequence complementarity with an appropriately tailored siRNA molecule.

Generally, siRNA is involved in the RNA interference (RNAi) pathway where it interferes with the expression of a specific gene. These siRNAs can bind to specific RNA molecules, resulting in an increase or decrease in the expression of a specific gene. Therefore, siRNAs can be effective therapeutic agents for the treatment of multiple disease states, for example, Parkinson's disease, Lou Gehrig's disease, viral infection, including HIV infection, type 2 diabetes, obesity, hypercholesterolemia, rheumatoid arthritis, and various types of cancer.

The prostate is one of the male reproductive organs found in the pelvis below the urinary bladder. It functions to produce and store seminal fluid which provides nutrients and fluids that are vital for the survival of sperm. Like many other tissues, the prostate glands are also prone to develop either malignant (cancerous) or benign (non-cancerous) tumors. The American Cancer Society predicted that over 230,000 men would be diagnosed with prostrate cancer and over 30,000 men would die from the disease in year 2005. In fact, prostate cancer is one of the most common male cancers in western societies, and is the second leading form of malignancy among American men. Current treatment methods for prostrate cancer include hormonal therapy, radiation therapy, surgery, chemotherapy, photodynamic therapy, and combination therapy. The selection of a treatment generally varies depending on the stage of the cancer. However, many of these treatments affect the quality of life of the patient, especially those men who are diagnosed with prostrate cancer over age 50. For example, the use of hormonal drugs is often accompanied by side effects such as osteoporosis and liver damage. Such side effects might be mitigated by the use of treatments that are more selective or specific to the tissue being responsible for the disease state, and avoid non-target tissues like the bones or the liver. As described herein, prostate specific membrane antigen (PSMA) represents a target for such selective or specific treatments.

PSMA is named largely due to its higher level of expression on prostate cancer cells; however, its particular function on prostate cancer cells remains unresolved. PSMA is over-expressed in the malignant prostate tissues when compared to other organs in the human body such as kidney, proximal small intestine, and salivary glands. Though PSMA is expressed in brain, that expression is minimal, and most ligands of PSMA are polar and are not capable of penetrating the blood brain barrier. PSMA is a type II cell surface membrane-bound glycoprotein with ˜110 kD molecular weight, including an intracellular segment (amino acids 1-18), a transmembrane domain (amino acids 19-43), and an extensive extracellular domain (amino acids 44-750). While the functions of the intracellular segment and the transmembrane domains are currently believed to be insignificant, the extracellular domain is involved in several distinct activities. PSMA plays a role in central nervous system, where it metabolizes N-acetyl-aspartyl glutamate (NAAG) into glutamic and N-acetyl aspartic acid. Accordingly, it is also sometimes referred to as an N-acetyl alpha linked acidic dipeptidase (NAALADase). PSMA is also sometimes referred to as a folate hydrolase I (FOLH I) or glutamate carboxypeptidase (GCP II) due to its role in the proximal small intestine where it removes γ-linked glutamate from poly-γ-glutamated folate and α-linked glutamate from peptides and small molecules.

PSMA also shares similarities with human transferrin receptor (TfR), because both PSMA and TfR are type II glycoproteins. More specifically, PSMA shows 54% and 60% homology to TfR1 and TfR2, respectively. However, though TfR exists only in dimeric form due to the formation of inter-strand sulfhydryl linkages, PSMA can exist in either dimeric or monomeric form.

Unlike many other membrane-bound proteins, PSMA undergoes rapid internalization into the cell in a similar fashion to cell surface bound receptors like vitamin receptors. PSMA is internalized through clathrin-coated pits and subsequently can either recycle to the cell surface or go to lysosomes. It has been suggested that the dimer and monomer form of PSMA are inter-convertible, though direct evidence of the interconversion is being debated. Even so, only the dimer of PSMA possesses enzymatic activity, and the monomer does not.

Though the activity of the PSMA on the cell surface of the prostate cells remains under investigation, it has been recognized by the inventors herein that PSMA represents a viable target for the selective and/or specific delivery of agents, including nucleotides. Importantly, Applicants have shown that expression of PSMA on prostate cancer cells can be exploited in vivo to specifically target nucleotides, such as siRNAs, to prostate cancer cells.

### SUMMARY OF THE INVENTION

It has been discovered that nucleotides that are conjugated to ligands capable of binding to prostate specific membrane antigen (PSMA) via a linker may be useful in selectively targeting prostate cancer cells, and related pathogenic cell populations expressing or over-expressing PSMA. PSMA is a cell surface protein that is internalized in a process analogous to endocytosis observed with cell surface receptors, such as vitamin receptors. Accordingly, it has been discovered that certain conjugates that include a linker having a predetermined length, and/or a predetermined diameter, and/or preselected functional groups along its length may be used to target prostate cancer cells with nucleotides.

In one illustrative embodiment of the invention, conjugates having the formula

B-L-N

are described wherein B is a prostate specific membrane antigen (PSMA) binding or targeting ligand, L is a linker, and N is a nucleotide. As used herein, the term nucleotide N collectively includes single- and double-stranded segments of DNA or RNA, siRNA, microRNA, methylated RNA, iRNA, and oligonucleotides, antisense molecules, and ribozymes, and the like, unless otherwise indicated. For example, in one illustrative configuration, the conjugate described herein is used to deliver an siRNA segment to a population of prostate cancer cells. Other configurations are also contemplated and described herein. It is to be understood that analogs and derivatives of each of the foregoing B, L, and N are also contemplated and described herein, and that when used herein, the terms B, L, and N collectively refer to such analogs and derivatives.

In one illustrative embodiment, the linker L may be a releasable or non-releasable linker. In one aspect, the linker L is at least about 7 atoms in length. In one variation, the linker L is at least about 10 atoms in length. In one variation, the linker L is at least about 14 atoms in length. In another variation, the linker is at least about 35 atoms in length. In another variation, the linker L is between about 7 and about 31, between about 7 and about 24, or between about 7 and about 20 atoms in length. In another variation, the linker L is between about 14 and about 31, between about 20 and about 46, between about 14 and about 24, or between about 14 and about 20 atoms in length.

In an alternative aspect, the linker L is at least about 10 angstroms (Å) in length. In one variation, the linker L is at least about 15 Å in length. In another variation, the linker L is at least about 20 Å in length. In another variation, the linker L is at least about 20 Å in length. In another variation, the linker L is in the range from about 10 Å to about 45 Å in length.

In an alternative aspect, at least a portion of the length of the linker L is about 5 Å in diameter or less at the end connected to the binding ligand B. In one variation, at least a portion of the length of the linker L is about 4 Å or less, or about 3 Å or less in diameter at the end connected to the binding ligand B. It is appreciated that the illustrative embodiments that include a diameter requirement of about 5 Å or less, about 4 Å or less, or about 3 Å or less may include that requirement for a predetermined length of the linker, thereby defining a cylindrical-like portion of the linker. Illustratively, in another variation, the linker includes a cylindrical portion at the end connected to the binding ligand that is at least about 7 Å in length and about 5 Å or less, about 4 Å or less, or about 3 Å or less in diameter.

In another embodiment, the linker L includes one or more hydrophilic linkers capable of interacting with one or more residues of PSMA, including amino acids that have hydrophilic side chains, such as Ser, Thr, Cys, Arg, Orn, Lys, Asp, Glu, Gln, and like residues. In another embodiment, the linker L includes one or more hydrophobic linkers capable of interacting with one or more residues of PSMA, including amino acids that have hydrophobic side chains, such as Val, Leu, Ile, Phe, Tyr, Met, and like residues. It is to be understood that the foregoing embodiments and aspects may be included in the linker L either alone or in combination with each other. For example, linkers L that are at least about 7 atoms in length and about 5 Å, about 4 Å or less, or about 3 Å or less in diameter or less are contemplated and described herein, and also include one or more hydrophobic linkers capable of interacting with one or more residues of PSMA, including Val, Leu, Ile, Phe, Tyr, Met, and like residues are contemplated and described herein.

In another embodiment, one end of the linker is not branched and comprises a chain of carbon, oxygen, nitrogen, and sulfur atoms. In one embodiment, the linear chain of carbon, oxygen, nitrogen, and sulfur atoms is at least 5 atoms in length. In one variation, the linear chain is at least 7 atoms, or at least 10 atoms in length. In another embodiment, the chain of carbon, oxygen, nitrogen, and sulfur atoms are not substituted. In one variation, a portion of the chain of carbon, oxygen, nitrogen, and sulfur atoms is cyclized with a divalent fragment. For example, a linker (L) comprising the dipeptide Phe-Phe may include a piperazin-1,4-diyl structure by cyclizing two nitrogens with an ethylene fragment, or substituted variation thereof.

In another embodiment, a composition comprising a prostate cancer cell targeting effective amount of the composition of any one of the conjugates described herein, and a component selected from the group consisting of carriers, diluents, and excipients, and combinations thereof is described.

In another embodiment, a method for specifically targeting prostate cancer cells in an animal, the method comprising the steps of administering to the animal an effective amount of a composition of any one of the conjugates or compounds described herein, optionally with a component selected from the group consisting of carriers, diluents, and excipients, and combinations thereof; and specifically targeting prostate cancer cells is described.

A method of reducing the expression of a gene in a cell using a PSMA ligand nucleotide conjugate, the method comprising the steps of providing a composition comprising any one of the conjugates or compounds described herein to the cell; wherein the composition binds to and is internalized into the cell; and reducing the expression of the gene is described.

In another embodiment, a method of treating a patient in need of relieve from prostate cancer, the method comprising the step of administering to the patient a composition comprising a therapeutically effective amount of any one of the compositions, compounds, or conjugates described herein is described.

In any embodiment, a process for preparing the compositions described herein, the process comprising the step of forming a thiol intermediate of the formula B-L′-SH or a thiol intermediate of the formula N-L′-SH;

and reacting the thiol intermediate with a compound of the formula B-L″ or N-L″ wherein L′ is a divalent linker; and L″ is a divalent linker comprising a thiol reactive group.

### BRIEF DESCRIPTION OF THE DRAWINGS

FIG. 1. Cell bound radioactivity versus concentration of SK28-^{99m}Tc (K_{d}=18.12 nM) in the presence (▴) or absence (▪) of excess PMPA.

FIG. 2. In Vitro Binding Studies Using LNCaP Cells and SK33 (14 atom linker). LNCaP cells containing increasing concentrations of DUPA-^{99m}Tc in the presence (▴) or absence (▪) of excess PMPA.

FIG. 3. Cell bound radioactivity verses concentration of SK28-^{99m}Tc; at 4° C. (▪) and at 37° C. (▴).

FIG. 4. Plot of cell bound radioactivity versus concentration of DUPA-Linker-⁹⁹Tc imaging agents: (▪) 0-atom linker (K_{d}=171 nM); (▴) 7-atom linker (K_{d}=68 nM); (▾) 14-atom linker (K_{d}=15 nM); (♦) 16-atom linker (K_{d}=40 nM).

FIG. 5. K_{D} values for DUPA-Linker-^{99m}Tc compounds binding to LNCaP cells.

FIG. 6. Images of LNCaP cells (a) not treated, (b) treated with DUPA-dsDNA-Cy5, (c) treated with and 100× PMPA.

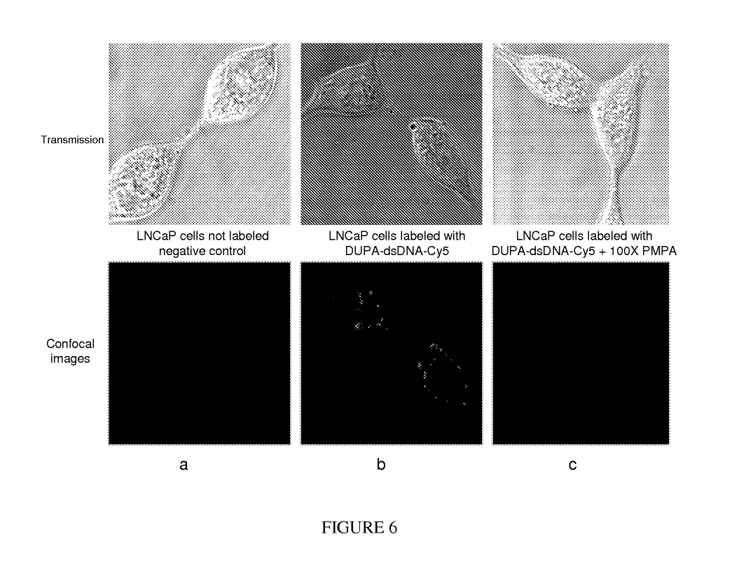

FIG. 7. Combined white light and fluorescent images of a nu/nu mouse with a tumor resulting from subcutaneous injection of LNCaP cells, treated with DUPA-dsDNA-Cy5. This result shows that cells that over express or selectively express PSMA can be selectively targeted by a siRNA-DUPA conjugate.

### DETAILED DESCRIPTION

The present invention relates to compounds, compositions, and methods for use in targeting nucleotides to prostate cancer cells. Methods of treating prostate cancer with the compounds and compositions described herein are also provided. Also provided are methods of preparing the compounds and compositions described herein. More particularly, the invention is directed to PSMA binding ligand conjugates for use in specifically targeting the conjugates to prostate cancer cells.

In one embodiment, the pathogenic cells that are specifically targeted using the PSMA binding ligand conjugates of the invention are prostate cancer cells. In various embodiments, the population of prostate cancer cells may be a cancer cell population that is tumorigenic, including benign tumors and malignant tumors, or it can be non-tumorigenic. The cancer cell population may arise spontaneously or by such processes as mutations present in the germline of the host animal or somatic mutations, or it may be chemically-, virally-, or radiation-induced.

In accordance with the invention, the phrases “specifically targeting”, “specific targeting”, and “specifically targeted” mean that the PSMA binding ligand conjugates described herein are preferentially targeted to prostate cancer cells that preferentially express or overexpress PSMA as evidenced by the ability to detect accumulation of the PSMA binding ligand conjugates in the specifically targeted cell type over accumulation in normal tissues that do not express the receptor for the ligand.

As used herein, the term “nucleotide” (N) includes an oligonucleotide, an iRNA, an siRNA, a microRNA, a ribozyme, an antisense molecule, or analogs or derivatives thereof. The nucleotide N can be RNA or DNA, or combinations thereof, and can be single or double-stranded. If the nucleotide N is double-stranded, the nucleotide N contains a sense strand and an antisense strand. If the nucleotide N is single-stranded, the strand is preferably an antisense strand. Typically, the nucleotide strands, if the nucleotide is double-stranded, are two separate molecules rather than two separate sequences on the same nucleotide strand. The PSMA binding ligand can be coupled to the sense strand or the antisense strand, or both.

In one embodiment, each strand of the nucleotide N includes about 15 to about 49 bases. In another embodiment, each strand of the nucleotide N includes about 19 to about 25 bases. In another embodiment, each strand of the nucleotide N includes about 15 to about 23 bases. In another embodiment, each strand of the nucleotide N includes about 21 to about 23 bases. In another embodiment, each strand of the nucleotide N includes about 21 to about 23 bases, with a duplex region of about 15 to about 23 base pairs. In another embodiment, the nucleotide N includes a single-stranded overhang at the 5′ and/or the 3′ end including about 2 to about 3 bases. Preferably, the single-stranded overhang is a 3′ overhang including about 2 to about 3 bases. In another embodiment, the nucleotide N is blunt-ended at least one end of the nucleotide. In another embodiment, the nucleotide N is a small interfering RNA, also referred to as siRNA.

In each of the forgoing, it is to be understood that nucleotide N may include not only natural bases, such as A, C, T, U, and G, but also may contain non-natural analogs and derivatives of such bases. For example, bases or analogs and derivatives of bases that may further stabilize the nucleotide against degradation (e.g., make the nucleotide nuclease resistant) or metabolism can be used. In another embodiment, other derivatives of the nucleotide N may be used, including 2′-F or 2′-OMe sugar modifications, 5-alkylamino or 5-allylamino base modifications, or other derivatives of naturally occurring bases, or phosphorothioate, P-alkyl, phosphonate, phosphoroselenate, or phosphoroamidate modifications of the nucleotide backbone or modifications of the backbone or a terminal phosphate with these or other phosphate analogs, or combinations thereof. The modifications can be made at any position in the nucleotide N, and can be any of the modifications described, for example, in WO 2009/082606, incorporated herein by reference. Methods of modifying nucleotides to stabilize nucleotides are well-known in the art. The nucleotide N described herein can be synthesized by methods well-known in the art such as those described in Trufert et al., Tetrahedron, 52:3005 (1996), Martin, Helv. Chim. Acta, 78, 486-504 (1995), or WO 2009/082606, each incorporated herein by reference.

In various illustrative embodiments, any nucleotide N (e.g., siRNA) that is complementary to the specific target gene of interest can be attached to a binding ligand as herein described.

The binding ligand (B) nucleotide delivery conjugates can be used to target prostate cancer cells in the host animal wherein the cells have an accessible binding site for the binding ligand (B), or analog or derivative thereof, wherein the binding site is uniquely expressed, overexpressed, or preferentially expressed by the prostate cancer cells. The specific targeting of the cells is mediated by the binding of the ligand moiety of the binding ligand (B) nucleotide delivery conjugate to a ligand receptor, transporter, or other surface-presented protein that specifically binds the binding ligand (B), or analog or derivative thereof, and which is uniquely expressed, overexpressed, or preferentially expressed by the prostate cancer cells. A surface-presented protein uniquely expressed, overexpressed, or preferentially expressed by the prostate cancer cells is a receptor not present or present at lower concentrations on non-prostate cancer cells providing a means for specific targeting of the prostate cancer cells.

In one embodiment, the nucleotide could be released by a protein disulfide isomerase inside the cell where a releasable linker is a disulfide group. The nucleotide may also be released by a hydrolytic mechanism, such as acid-catalyzed hydrolysis, as described for certain beta elimination mechanisms, or by an anchimerically assisted cleavage through an oxonium ion or lactonium ion producing mechanism. The selection of the releasable linker or linkers will dictate the mechanism by which the nucleotide is released from the conjugate. It is appreciated that such a selection can be pre-defined by the conditions wherein the nucleotide conjugate will be used. Alternatively, the PSMA binding ligand conjugates can be internalized into the targeted cells upon binding, and the PSMA binding ligand and the nucleotide can remain associated intracellularly with the nucleotide exhibiting its effects without dissociation from the ligand.

In one embodiment, the nucleotides for use in the methods described herein remain stable in serum for at least 4 hours. In another embodiment the nucleotides have an IC₅₀ in the nanomolar range, and, in another embodiment, the nucleotides are water soluble. If the nucleotide is not water soluble, the linkers (L) described herein can be derivatized to enhance water solubility. Nucleotide analogs or derivatives can also be used, such as methylated bases to enhance stability of the nucleotide.

Additionally, more than one type of PSMA binding ligand conjugate can be used. Illustratively, for example, cells of the host animal can be targeted with conjugates with different PSMA binding ligands, but the same nucleotide. In other embodiments, the host animal cells can be targeted with conjugates comprising the same PSMA binding ligand linked to different nucleotides, or various PSMA binding ligands linked to various nucleotides.

In one embodiment, a method of treating a patient harboring a population of prostate cancer cells is provided. The method comprises the step of administering to the patient a composition comprising a therapeutically effective amount of any of the PSMA binding ligand nucleotide conjugates described herein. In another illustrative embodiment, a method of specifically targeting a nucleotide to prostate cancer cells in a host animal is provided. The method comprises the step of administering any of the PSMA binding ligand nucleotide conjugates described herein to the animal where the prostate cancer cells overexpress or selectively expresses a receptor for the ligand.

In another illustrative embodiment, a method is provided of reducing the expression of a gene in a prostate cancer cell using a PSMA binding ligand nucleotide conjugate. The method comprises the step of providing the PSMA binding ligand conjugate of the invention to the cell wherein the conjugate binds to and is internalized into the cell, and wherein expression of the gene is reduced. In one embodiment, the reduction in expression of the gene is complete and in another embodiment, the reduction in expression of the gene is partial. In this embodiment of the invention, gene expression can be reduced in vitro, such as in a cell type (e.g., primary cells) or a cell line (e.g., a transformed cell line) or in vivo, such as in an animal or a human or in a tissue. In one illustrative embodiment, the reduction of expression occurs in vitro and the reduction in expression occurs in a cell that has been genetically modified using molecular biology techniques. Such techniques are described in Sambrook et al., “Molecular Cloning: A Laboratory Manual”, 3rd Edition, Cold Spring Harbor Laboratory Press, (2001), incorporated herein by reference. In one embodiment, the reduction in gene expression that occurs in vitro or in vivo can be reduction in expression of a reporter gene, such as β-galactosidase, green fluorescent protein, or luciferase.

In another embodiment, a process for preparing any of the PSMA binding ligand nucleotide conjugates described herein is provided. The process comprises the step of forming a thiol intermediate of the formula B-L′-SH or a thiol intermediate of the formula N-L′-SH, and reacting the thiol intermediate with a compound of the formula B-L″ or N-L″ wherein L′ is a divalent linker, and L″ is a divalent linker comprising a thiol reactive group.

In yet another embodiment, a kit is provided. The kit can comprise a container, a composition comprising any of the PSMA binding ligand nucleotide conjugates described herein, a sterile package containing the composition, and instructions for use.

Nucleotide delivery conjugates are described herein where a PSMA binding ligand is attached to a releasable or non-releasable linker which is attached to a nucleotide.

Illustratively, the bivalent linkers described herein may be included in linkers used to prepare PSMA-binding nucleotide conjugates of the following formula:

B-L-N

where B is a PSMA-binding moiety, including analogs or derivatives thereof, L is a linker, N is an nucleotide, including analogs or derivatives thereof. The linker L can comprise multiple bivalent linkers, including the bivalent linkers described herein. It is also to be understood that as used herein, D collectively refers to nucleotides, and analogs and derivatives thereof.

The linker may also include one or more spacer linkers and optionally additional releasable linkers. The spacer and releasable linkers may be attached to each other in any order or combination. Similarly, the PSMA binding ligand may be attached to a spacer linker or to a releasable linker. Similarly, the nucleotide may be attached to a spacer linker or to a releasable linker. Each of these components of the conjugates may be connected through existing or additional heteroatoms on the targeting ligand, nucleotide, releasable or spacer linker. Illustrative heteroatoms include nitrogen, oxygen, sulfur, and the formulae —(NHR¹NHR²)—, —SO—, —(SO₂)—, and —N(R³)O—, wherein R¹, R², and R³ are each independently selected from hydrogen, alkyl, heteroalkyl, heterocyclyl, aryl, heteroaryl, arylalkyl, heteroarylalkyl, and the like, each of which may be optionally substituted.

In one illustrative embodiment, compounds are described herein that include linkers having predetermined length and diameter dimensions. In one aspect, linkers are described herein that satisfy one or more minimum length requirements, or a length requirement falling within a predetermined range. In another aspect, satisfaction of a minimum length requirement may be understood to be determined by computer modeling of the extended conformations of linkers. In another aspect, satisfaction of a minimum length requirement may be understood to be determined by having a certain number of atoms, whether or not substituted, forming a backbone chain of atoms connecting the binding ligand (B) with the nucleotide (N). In another embodiment, the backbone chain of atoms is cyclized with another divalent fragment. In another aspect, linkers are described herein that satisfy one or more maximum or minimum diameter requirements. In another aspect, satisfaction of a maximum or minimum diameter requirement may be understood to be determined by computer modeling of various conformations of linkers modeled as the space-filling, CPK, or like configurations. In another aspect, satisfaction of a maximum or minimum diameter requirement may be understood to be apply to one or more selected portions of the linker, for example the portion of the linker proximal to the binding ligand (B), or the portion of the linker proximal to the nucleotide (N), and the like. In another aspect, linkers are described herein that satisfy one or more chemical composition requirements, such as linkers that include one or more polar groups that may positively interact with the one or more Arg or Lys side-chain nitrogens and/or Asp or Glu side chain oxygens found in the funnel portion of PSMA. In one variation, linkers are described herein that satisfy one or more chemical composition requirements, such as linkers that include one or more non-polar groups that may positively interact with the one or more Tyr or Phe side-chain carbons found in the funnel portion of PSMA.

In one embodiment, the atom-length of the linker is defined by the number of atoms separating the binding or targeting ligand B, or analog or derivative thereof, and the nucleotide N, or analog or derivative thereof. Accordingly, in configurations where the binding ligand B, or analog or derivative thereof, is attached directly to the nucleotide N, or analog or derivative thereof, the attachment is also termed herein as a “0-atom” linker. It is understood that such 0-atom linkers include the configuration wherein B and N are directly attached by removing a hydrogen atom from each attachment point on B and N, respectively. It is also understood that such 0-atom linkers include the configuration wherein B and N are attached through an overlapping heteroatom by removing a hydrogen atom from one of B or N, and a heteroatom functional group, such as OH, SH, NH₂, and the like from the other of B or N. It is also understood that such 0-atom linkers include the configuration wherein B and N are attached through a double bond, which may be formed by removing two hydrogen atoms from each attachment point on B and N, respectively, or whereby B and N are attached through one or more overlapping heteroatoms by removing two hydrogen atoms, one hydrogen and one heteroatom functional group, or two heteroatom functional groups, such as OH, SH, NH₂, and the like, from each of B or N. In addition, B and N may be attached through a double bond formed by removing a double bonded heteroatom functional group, such as O, S, NH, and the like, from one or both of B or N. It is also to be understood that such heteroatom functional groups include those attached to saturated carbon atoms, unsaturated carbon atoms (including carbonyl groups), and other heteroatoms. Similarly, the length of linkers that are greater than 0 atoms are defined in an analogous manner.

Accordingly, in another illustrative embodiment, linkers (L) are described having a chain length of at least 7 atoms. In one variation, linkers (L) are described having a chain length of at least 14 atoms. In another variation, linkers (L) are described having a chain length in the range from about 7 atoms to about 20 atoms. In another variation, linkers (L) are described having a chain length in the range from about 14 atoms to about 24 atoms.

In another embodiment, the length of the linker (L) is defined by measuring the length of an extended conformation of the linker. Such extended conformations may be measured in art-recognized computer modeling programs, such as PC Model 7 (MMX). Accordingly, in another illustrative embodiment, linkers are described having a chain length of at least 15 Å, at least 20 Å, or at least 25 Å.

In another embodiment, linkers are described having at least one hydrophobic side chain group, such as an alkyl, cycloalkyl, aryl, arylalkyl, or like group, each of which is optionally substituted. In one aspect, the hydrophobic group is included in the linker by incorporating one or more Phe or Tyr groups, including substituted variants thereof, and analogs and derivatives thereof, in the linker chain. It is appreciated that such Phe and/or Tyr side chain groups may form positive pi-pi (π-π) interactions with Tyr and Phe residues found in the funnel of PSMA. In addition, it is appreciated that the presence of large side chain branches, such as the arylalkyl groups found on Phe and Tyr may provide a level of conformational rigidity to the linker, thus limiting the degrees of freedom, and reducing coiling and promoting extended conformations of the linker. Without being bound by theory, it is appreciated that such entropy restrictions may increase the overall binding energy of the bound conjugates described herein. In addition, it is appreciated that the rigidity increases that may be provided by sterically hindered side chains, such as Phe and Tyr described herein, may reduce or prevent coiling and interactions between the ligand and the nucleotide. It has been discovered herein that the funnel shaped tunnel leading to the catalytic site or active site of PSMA imposes length, shape, and/or chemical composition requirements on the linker portion of conjugates of PSMA binding ligands and nucleotides that positively and negatively affect the interactions between PSMA and those conjugates. Described herein are illustrative embodiments of those conjugates that include such length, shape, and/or chemical composition requirements on the linker. Such length, shape, and/or chemical composition requirements were assessed using molecular modeling. For example, the space filling and surface model of the PSMA complex with (s)-2-(4-iodobenzensylphosphonomethyl)-pentanedioic [2-PMPA derivative] PDB ID code 2C6P were generated using PROTEIN EXPLORER. The PROTEIN EXPLORER model verified the 20 Å deep funnel, and also showed diameter features at various locations along the funnel that may be used to define linkers having favorable structural features. In addition, the model showed that close to the active site of PSMA, there are a higher number of hydrophobic residues that may provide additional binding interactions when the corresponding functional groups are included in the linker. Finally, the model showed the presence of three hydrophobic pockets that may provide additional binding interactions when the corresponding functional groups are included in the linker.

In another illustrative embodiment, the following molecular models were created for a conjugate of MUPA and a tripeptide ^{99m}Tc imaging agent connected by a 9-atom linker and syn-SK33 including a branched 14-atom linker. The models were created using PC Model 7 (MMX) with energy minimization, and using the following bond length parameters: C—C (sp³−sp³)=1.53 Å, C—C (sp³−sp²)=1.51 Å, C—N (sp³−N)=1.47 Å, C—N (sp²−N)=1.38 Å. Such models may be used to calculate the length of the linker connecting the binding ligand (B) and the nucleotide (N). In addition, such models may be modified to create extended conformations, and subsequently used to calculate the length of the linker connecting the binding ligand (B) and the nucleotide (N).

The first human PSMA gene was cloned from LNCaP cells and is reported to be located in chromosome 11p11-12. In addition, there is a PSMA-like gene located at the loci 11q14.3. The crystal structure of PSMA has been reported by two different groups at different resolutions, and each shows that the active site contains two zinc atoms, confirming that PSMA is also considered a zinc metalloprotease. Davis et al, PNAS, 102:5981-86, (2005) reported the crystal structure at low resolution (3.5 Å), while Mesters et al, The EMBO Journal, 1-10 (2006) reported the crystal structure at higher resolution (2-2.2 Å), the disclosures of which are incorporated herein by reference, in addition, the entire disclosure of any document referenced herein is also incorporated by reference in its entirety. The crystal structures show that PSMA is a homodimer that contains a protease domain, an apical domain, a helical domain and a CPG2 dimerization domain. The protease domain of PSMA contains a binuclear zinc site, catalytic residues and a substrate binding region including three arginine residues (also referred to as a substrate binding arginine patch). In the crystal structure, the two zinc ions in the active site are each ligated to an oxygen of phosphate, or to the phosphinate moiety of the inhibitor GPI 18431 for the co-crystal structure. In the high resolution crystal structures of the extracelluar domain, PSMA was co-crystallized with potent inhibitors, weak inhibitors, and glutamate at 2.0, 2.4, and 2.2 Å, respectively. The high resolution crystal structure shows a 20 Å deep funnel shaped tunnel leads to the catalytic site or active site of PSMA. The funnel is lined with the side chains of a number of Arg and Lys residues, Asp and Glu residues, and Tyr and Phe residues.

In another embodiment, the linker (L) is a chain of atoms selected from C, N, O, S, Si, and P. The linker may have a wide variety of lengths, such as in the range from about 7 to about 100. The atoms used in forming the linker may be combined in all chemically relevant ways, such as chains of carbon atoms forming alkylene groups, chains of carbon and oxygen atoms forming polyoxyalkylene groups, chains of carbon and nitrogen atoms forming polyamines, and others. In addition, it is to be understood that the bonds connecting atoms in the chain may be either saturated or unsaturated, such that for example, alkanes, alkenes, alkynes, cycloalkanes, arylenes, imides, and the like may be divalent radicals that are included in the linker. In addition, it is to be understood that the atoms forming the linker may also be cyclized upon each other to form divalent cyclic radicals in the linker. In each of the foregoing and other linkers described herein the chain forming the linker may be substituted with a wide variety of groups.

In another embodiment, linkers (L) are described that include at least one releasable linker. In one variation, linkers (L) are described that include at least two releasable linkers. In another variation, linkers (L) are described that include at least one self-immolative linker. In another variation, linkers (L) are described that include at least one releasable linker that is not a disulfide. In another embodiment, linkers (L) are described that do not include a releasable linker.

It is appreciated that releasable linkers may be used when the nucleotide to be delivered is advantageously liberated from the binding ligand-linker conjugate so that the free nucleotide will have the same or nearly the same effect at the target as it would when administered without the targeting provided by the conjugates described herein. In another embodiment, the linker L is a non-releasable linker. It is appreciated that non-releasable linkers may be used when the nucleotide is advantageously retained by the binding ligand-linker conjugate. It is to be understood that the choice of a releasable linker or a non-releasable linker may be made independently for each application or configuration of the conjugates, without limiting the invention described herein. It is to be further understood that the linkers L described herein comprise various atoms, chains of atoms, functional groups, and combinations of functional groups. Where appropriate in the present disclosure, the linker L may be referred to by the presence of spacer linkers, releasable linkers, and heteroatoms. However, such references are not to be construed as limiting the definition of the linkers L described herein.

The linker (L) comprising spacer and/or releasable linkers (i.e., cleavable linkers) can be any biocompatible linker. The releasable or cleavable linker can be, for example, a linker susceptible to cleavage under the reducing or oxidizing conditions present in or on cells, a pH-sensitive linker that may be an acid-labile or base-labile linker, or a linker that is cleavable by biochemical or metabolic processes, such as an enzyme-labile linker. In one embodiment, the spacer and/or releasable linker comprises about 1 to about 30 atoms, or about 2 to about 20 atoms. Lower molecular weight linkers (i.e., those having an approximate molecular weight of about 30 to about 300) are also described. Precursors to such linkers may be selected to have either nucleophilic or electrophilic functional groups, or both, optionally in a protected form with a readily cleavable protecting group to facilitate their use in synthesis of the intermediate species.

The term “releasable linker” as used herein refers to a linker that includes at least one bond that can be broken under physiological conditions (e.g., a pH-labile, acid-labile, oxidatively-labile, or enzyme-labile bond). The cleavable bond or bonds may be present in the interior of a cleavable linker and/or at one or both ends of a cleavable linker. It should be appreciated that such physiological conditions resulting in bond breaking include standard chemical hydrolysis reactions that occur, for example, at physiological pH, or as a result of compartmentalization into a cellular organelle such as an endosome having a lower pH than cytosolic pH. Illustratively, the bivalent linkers described herein may undergo cleavage under other physiological or metabolic conditions, such as by the action of a glutathione mediated mechanism. It is appreciated that the lability of the cleavable bond may be adjusted by including functional groups or fragments within the bivalent linker L that are able to assist or facilitate such bond breakage, also termed anchimeric assistance. The lability of the cleavable bond can also be adjusted by, for example, substitutional changes at or near the cleavable bond, such as including alpha branching adjacent to a cleavable disulfide bond, increasing the hydrophobicity of substituents on silicon in a moiety having a silicon-oxygen bond that may be hydrolyzed, homologating alkoxy groups that form part of a ketal or acetal that may be hydrolyzed, and the like. In addition, it is appreciated that additional functional groups or fragments may be included within the bivalent linker L that are able to assist or facilitate additional fragmentation of the PSMA binding nucleotide linker conjugates after bond breaking of the releasable linker.

In another embodiment, the linker includes radicals that form one or more spacer linkers and/or releasable linkers that are taken together to form the linkers described herein having certain length, diameter, and/or functional group requirements.

Another illustrative embodiment of the linkers described herein, include releasable linkers that cleave under the conditions described herein by a chemical mechanism involving beta elimination. In one aspect, such releasable linkers include beta-thio, beta-hydroxy, and beta-amino substituted carboxylic acids and derivatives thereof, such as esters, amides, carbonates, carbamates, and ureas. In another aspect, such releasable linkers include 2- and 4-thioarylesters, carbamates, and carbonates.

It is to be understood that releasable linkers may also be referred to by the functional groups they contain, illustratively such as disulfide groups, ketal groups, and the like, as described herein. Accordingly, it is understood that a cleavable bond can connect two adjacent atoms within the releasable linker and/or connect other linkers, or the binding ligand B, or the nucleotide N, as described herein, at either or both ends of the releasable linker. In the case where a cleavable bond connects two adjacent atoms within the releasable linker, following breakage of the bond, the releasable linker is broken into two or more fragments. Alternatively, in the case where a cleavable bond is between the releasable linker and another moiety, such as an additional heteroatom, a spacer linker, another releasable linker, the nucleotide N, or analog or derivative thereof, or the binding ligand B, or analog or derivative thereof, following breakage of the bond, the releasable linker is separated from the other moiety.

In another embodiment, the releasable and spacer linkers may be arranged in such a way that subsequent to the cleavage of a bond in the bivalent linker, released functional groups anchimerically assist the breakage or cleavage of additional bonds, as described above. An illustrative embodiment of such a bivalent linker or portion thereof includes compounds having the formula:

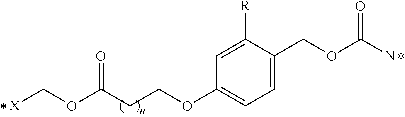

where X is an heteroatom, such as nitrogen, oxygen, or sulfur, n is an integer selected from 0, 1, 2, and 3, R is hydrogen, or a substituent, including a substituent capable of stabilizing a positive charge inductively or by resonance on the aryl ring, such as alkoxy, and the like, and the symbol (*) indicates points of attachment for additional spacer or releasable linkers, or heteroatoms, forming the bivalent linker, or alternatively for attachment of the nucleotide, or analog or derivative thereof, or the PSMA binding ligand, or analog or derivative thereof. It is appreciated that other substituents may be present on the aryl ring, the benzyl carbon, the alkanoic acid, or the methylene bridge, including but not limited to hydroxy, alkyl, alkoxy, alkylthio, halo, and the like. Assisted cleavage may include mechanisms involving benzylium intermediates, benzyne intermediates, lactone cyclization, oxonium intermediates, beta-elimination, and the like. It is further appreciated that, in addition to fragmentation subsequent to cleavage of the releasable linker, the initial cleavage of the releasable linker may be facilitated by an anchimerically assisted mechanism.

In this embodiment, the hydroxyalkanoic acid, which may cyclize, facilitates cleavage of the methylene bridge, by for example an oxonium ion, and facilitates bond cleavage or subsequent fragmentation after bond cleavage of the releasable linker. Alternatively, acid catalyzed oxonium ion-assisted cleavage of the methylene bridge may begin a cascade of fragmentation of this illustrative bivalent linker, or fragment thereof. Alternatively, acid-catalyzed hydrolysis of the carbamate may facilitate the beta elimination of the hydroxyalkanoic acid, which may cyclize, and facilitate cleavage of methylene bridge, by for example an oxonium ion. It is appreciated that other chemical mechanisms of bond breakage or cleavage under the metabolic, physiological, or cellular conditions described herein may initiate such a cascade of fragmentation. It is appreciated that other chemical mechanisms of bond breakage or cleavage under the metabolic, physiological, or cellular conditions described herein may initiate such a cascade of fragmentation.

Illustrative mechanisms for cleavage of the bivalent linkers described herein include the following 1,4 and 1,6 fragmentation mechanisms

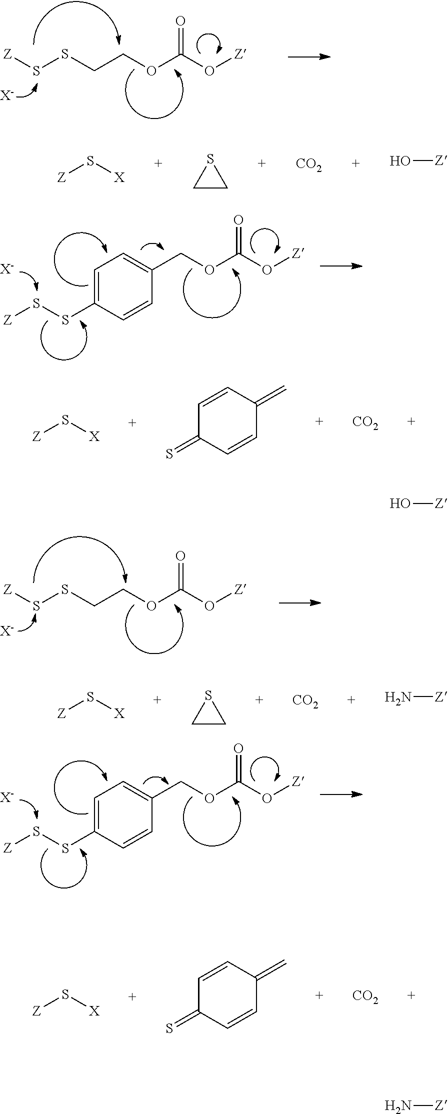

where X is an exogenous or endogenous nucleophile, glutathione, or bioreducing agent, and the like, and either of Z or Z′ is a PSMA binding ligand, or a nucleotide, or either of Z or Z′ is a PSMA binding ligand, or a nucleotide connected through other portions of the bivalent linker. It is to be understood that although the above fragmentation mechanisms are depicted as concerted mechanisms, any number of discrete steps may take place to effect the ultimate fragmentation of the bivalent linker to the final products shown. For example, it is appreciated that the bond cleavage may also occur by acid catalyzed elimination of the carbamate moiety, which may be anchimerically assisted by the stabilization provided by either the aryl group of the beta sulfur or disulfide illustrated in the above examples. In those variations of this embodiment, the releasable linker is the carbamate moiety. Alternatively, the fragmentation may be initiated by a nucleophilic attack on the disulfide group, causing cleavage to form a thiolate. The thiolate may intermolecularly displace a carbonic acid or carbamic acid moiety and form the corresponding thiacyclopropane. In the case of the benzyl-containing bivalent linkers, following an illustrative breaking of the disulfide bond, the resulting phenyl thiolate may further fragment to release a carbonic acid or carbamic acid moiety by forming a resonance stabilized intermediate. In any of these cases, the releaseable nature of the illustrative bivalent linkers described herein may be realized by whatever mechanism may be relevant to the chemical, metabolic, physiological, or biological conditions present.

Other illustrative mechanisms for bond cleavage of the releasable linker include oxonium-assisted cleavage as follows:

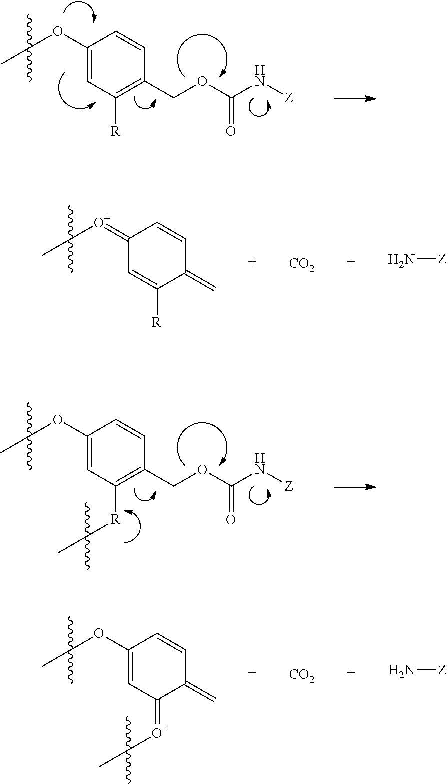

where Z is the PSMA binding ligand, or analog or derivative thereof, or the nucleotide, or analog or derivative thereof, or each is a PSMA binding ligand or nucleotide moiety in conjunction with other portions of the polyvalent linker, such as a nucleotide or PSMA binding ligand moiety including one or more spacer linkers and/or other releasable linkers. In this embodiment, acid-catalyzed elimination of the carbamate leads to the release of CO₂ and the nitrogen-containing moiety attached to Z, and the formation of a benzyl cation, which may be trapped by water, or any other Lewis base.

In one embodiment, the releasable linker includes a disulfide.

In another embodiment, the releasable linker may be a divalent radical comprising alkyleneaziridin-1-yl, alkylenecarbonylaziridin-1-yl, carbonylalkylaziridin-1-yl, alkylenesulfoxylaziridin-1-yl, sulfoxylalkylaziridin-1-yl, sulfonylalkylaziridin-1-yl, or alkylenesulfonylaziridin-1-yl, wherein each of the releasable linkers is optionally substituted with a substituent X², as defined below.

Additional illustrative releasable linkers include methylene, 1-alkoxyalkylene, 1-alkoxycycloalkylene, 1-alkoxyalkylenecarbonyl, 1-alkoxycycloalkylenecarbonyl, carbonylarylcarbonyl, carbonyl(carboxyaryl)carbonyl, carbonyl(biscarboxyaryl)carbonyl, haloalkylenecarbonyl, alkylene(dialkylsilyl), alkylene(alkylarylsilyl), alkylene(diarylsilyl), (dialkylsilyl)aryl, (alkylarylsilyl)aryl, (diarylsilyl)aryl, oxycarbonyloxy, oxycarbonyloxyalkyl, sulfonyloxy, oxysulfonylalkyl, iminoalkylidenyl, carbonylalkylideniminyl, iminocycloalkylidenyl, carbonylcycloalkylideniminyl, alkylenethio, alkylenearylthio, and carbonylalkylthio, wherein each of the releasable linkers is optionally substituted with a substituent X², as defined below.

In the preceding embodiment, the releasable linker may include oxygen, and the releasable linkers can be methylene, 1-alkoxyalkylene, 1-alkoxycycloalkylene, 1-alkoxyalkylenecarbonyl, and 1-alkoxycycloalkylenecarbonyl, wherein each of the releasable linkers is optionally substituted with a substituent X², as defined below, and the releasable linker is bonded to the oxygen to form an acetal or ketal. Alternatively, the releasable linker may include oxygen, and the releasable linker can be methylene, wherein the methylene is substituted with an optionally-substituted aryl, and the releasable linker is bonded to the oxygen to form an acetal or ketal. Further, the releasable linker may include oxygen, and the releasable linker can be sulfonylalkyl, and the releasable linker is bonded to the oxygen to form an alkylsulfonate.

In another embodiment of the above releasable linker embodiment, the releasable linker may include nitrogen, and the releasable linkers can be iminoalkylidenyl, carbonylalkylideniminyl, iminocycloalkylidenyl, and carbonylcycloalkylideniminyl, wherein each of the releasable linkers is optionally substituted with a substituent X², as defined below, and the releasable linker is bonded to the nitrogen to form an hydrazone. In an alternate configuration, the hydrazone may be acylated with a carboxylic acid derivative, an orthoformate derivative, or a carbamoyl derivative to form various acylhydrazone releasable linkers.

Alternatively, the releasable linker may include oxygen, and the releasable linkers can be alkylene(dialkylsilyl), alkylene(alkylarylsilyl), alkylene(diarylsilyl), (dialkylsilyl)aryl, (alkylarylsilyl)aryl, and (diarylsilyl)aryl, wherein each of the releasable linkers is optionally substituted with a substituent X², as defined below, and the releasable linker is bonded to the oxygen to form a silanol.

In the above releasable linker embodiment, the nucleotide can include a nitrogen atom, the releasable linker may include nitrogen, and the releasable linkers can be carbonylarylcarbonyl, carbonyl(carboxyaryl)carbonyl, carbonyl(biscarboxyaryl)carbonyl, and the releasable linker can be bonded to the heteroatom nitrogen to form an amide, and also bonded to the nucleotide nitrogen to form an amide.

In the above releasable linker embodiment, the nucleotide can include an oxygen atom, the releasable linker may include nitrogen, and the releasable linkers can be carbonylarylcarbonyl, carbonyl(carboxyaryl)carbonyl, carbonyl(biscarboxyaryl)carbonyl, and the releasable linker can form an amide, and also bonded to the nucleotide oxygen to form an ester.

The substituents X² can be alkyl, alkoxy, alkoxyalkyl, hydroxy, hydroxyalkyl, amino, aminoalkyl, alkylaminoalkyl, dialkylaminoalkyl, halo, haloalkyl, sulfhydrylalkyl, alkylthioalkyl, aryl, substituted aryl, arylalkyl, substituted arylalkyl, heteroaryl, substituted heteroaryl, carboxy, carboxyalkyl, alkyl carboxylate, alkyl alkanoate, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides. In this embodiment the releasable linker can include nitrogen, and the substituent X² and the releasable linker can form an heterocycle.

The heterocycles can be pyrrolidines, piperidines, oxazolidines, isoxazolidines, thiazolidines, isothiazolidines, pyrrolidinones, piperidinones, oxazolidinones, isoxazolidinones, thiazolidinones, isothiazolidinones, and succinimides.

In one embodiment, the polyvalent linkers described herein are or include compounds of the following formulae:

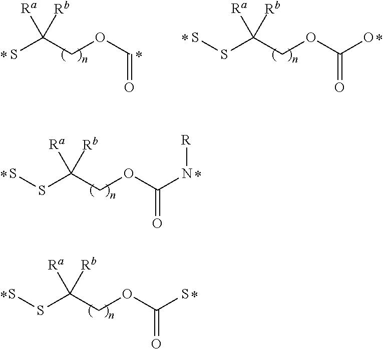

where n is an integer selected from 1 to about 4; R^{a} and R^{b} are each independently selected from the group consisting of hydrogen and alkyl, including lower alkyl such as C₁-C₄ alkyl that are optionally branched; or R^{a} and R^{b} are taken together with the attached carbon atom to form a carbocyclic ring; R is an optionally substituted alkyl group, an optionally substituted acyl group, or a suitably selected nitrogen protecting group; and (*) indicates points of attachment for the nucleotide, PSMA binding ligand, other polyvalent linkers, or other parts of the conjugate.

In another embodiment, the polyvalent linkers described herein are or include compounds of the following formulae

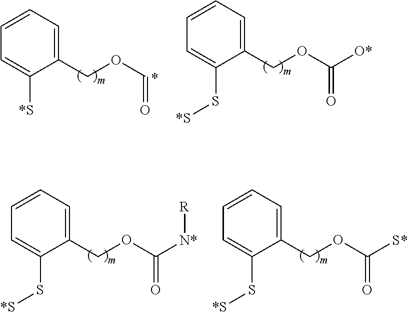

where m is an integer selected from 1 to about 4; R is an optionally substituted alkyl group, an optionally substituted acyl group, or a suitably selected nitrogen protecting group; and (*) indicates points of attachment for the nucleotide, PSMA binding ligand, other polyvalent linkers, or other parts of the conjugate.

In another embodiment, the polyvalent linkers described herein are or include compounds of the following formulae

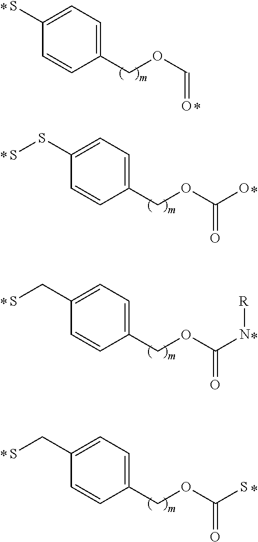

where m is an integer selected from 1 to about 4; R is an optionally substituted alkyl group, an optionally substituted acyl group, or a suitably selected nitrogen protecting group; and (*) indicates points of attachment for the nucleotide, PSMA binding ligand, imaging agent, diagnostic agent, other polyvalent linkers, or other parts of the conjugate.

In another embodiment, the linker L includes one or more spacer linkers. Such spacer linkers can be 1-alkylenesuccinimid-3-yl, optionally substituted with a substituent X¹, as defined below, and the releasable linkers can be methylene, 1-alkoxyalkylene, 1-alkoxycycloalkylene, 1-alkoxyalkylenecarbonyl, 1-alkoxycycloalkylenecarbonyl, wherein each of the releasable linkers is optionally substituted with a substituent X², as defined below, and wherein the spacer linker and the releasable linker are each bonded to the spacer linker to form a succinimid-1-ylalkyl acetal or ketal.

The spacer linkers can be carbonyl, thionocarbonyl, alkylene, cycloalkylene, alkylenecycloalkyl, alkylenecarbonyl, cycloalkylenecarbonyl, carbonylalkylcarbonyl, 1-alkylenesuccinimid-3-yl, 1-(carbonylalkyl)succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl)succinimid-3-yl, and 1-(carbonyltetrahydrofuranyl)succinimid-3-yl, wherein each of the spacer linkers is optionally substituted with a substituent X¹, as defined below. In this embodiment, the spacer linker may include an additional nitrogen, and the spacer linkers can be alkylenecarbonyl, cycloalkylenecarbonyl, carbonylalkylcarbonyl, 1-(carbonylalkyl)succinimid-3-yl, wherein each of the spacer linkers is optionally substituted with a substituent X¹, as defined below, and the spacer linker is bonded to the nitrogen to form an amide. Alternatively, the spacer linker may include an additional sulfur, and the spacer linkers can be alkylene and cycloalkylene, wherein each of the spacer linkers is optionally substituted with carboxy, and the spacer linker is bonded to the sulfur to form a thiol. In another embodiment, the spacer linker can include sulfur, and the spacer linkers can be 1-alkylenesuccinimid-3-yl and 1-(carbonylalkyl)succinimid-3-yl, and the spacer linker is bonded to the sulfur to form a succinimid-3-ylthiol.

In an alternative to the above-described embodiments, the spacer linker can include nitrogen, and the releasable linker can be a divalent radical comprising alkyleneaziridin-1-yl, carbonylalkylaziridin-1-yl, sulfoxylalkylaziridin-1-yl, or sulfonylalkylaziridin-1-yl, wherein each of the releasable linkers is optionally substituted with a substituent X², as defined below. In this alternative embodiment, the spacer linkers can be carbonyl, thionocarbonyl, alkylenecarbonyl, cycloalkylenecarbonyl, carbonylalkylcarbonyl, 1-(carbonylalkyl)succinimid-3-yl, wherein each of the spacer linkers is optionally substituted with a substituent X¹, as defined below, and wherein the spacer linker is bonded to the releasable linker to form an aziridine amide.

The substituents X¹ can be alkyl, alkoxy, alkoxyalkyl, hydroxy, hydroxyalkyl, amino, aminoalkyl, alkylaminoalkyl, dialkylaminoalkyl, halo, haloalkyl, sulfhydrylalkyl, alkylthioalkyl, aryl, substituted aryl, arylalkyl, substituted arylalkyl, heteroaryl, substituted heteroaryl, carboxy, carboxyalkyl, alkyl carboxylate, alkyl alkanoate, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides. In this embodiment the spacer linker can include nitrogen, and the substituent X¹ and the spacer linker to which they are bound to form an heterocycle.

Additional illustrative spacer linkers include alkylene-amino-alkylenecarbonyl, alkylene-thio-(carbonylalkylsuccinimid-3-yl), and the like, as further illustrated by the following formulae:

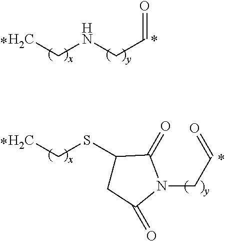

where the integers x and y are 1, 2, 3, 4, or 5:

In another embodiment, linkers that include hydrophilic regions are also described. In one aspect, the hydrophilic region of the linker forms part or all of a spacer linker included in the conjugates described herein. Illustrative hydrophilic spacer linkers are described in PCT international application serial No. PCT/US2008/068093, filed Jun. 25, 2008, the disclosure of which is incorporated herein by reference.

The term “cycloalkyl” as used herein includes molecular fragments or radicals comprising a bivalent chain of carbon atoms, a portion of which forms a ring. It is to be understood that the term cycloalkyl as used herein includes fragments and radicals attached at either ring atoms or non-ring atoms, such as, such as cyclopropyl, cyclohexyl, 3-ethylcyclopent-1-yl, cyclopropylethyl, cyclohexylmethyl, and the like.

The term “cycloalkylene” as used herein includes molecular fragments or radicals comprising a bivalent chain of carbon atoms, a portion of which forms a ring. It is to be understood that the term cycloalkyl as used herein includes fragments and radicals attached at either ring atoms or non-ring atoms, such as cycloprop-1,1-diyl, cycloprop-1,2-diyl, cyclohex-1,4-diyl, 3-ethylcyclopent-1,2-diyl, 1-methylenecyclohex-4-yl, and the like.

The terms “heteroalkyl” and “heteroalkylene” as used herein includes molecular fragments or radicals comprising monovalent and divalent, respectively, groups that are formed from a linear or branched chain of carbon atoms and heteroatoms, wherein the heteroatoms are selected from nitrogen, oxygen, and sulfur, such as alkoxyalkyl, alkyleneoxyalkyl, aminoalkyl, alkylaminoalkyl, alkyleneaminoalkyl, alkylthioalkyl, alkylenethioalkyl, alkoxyalkylaminoalkyl, alkylaminoalkoxyalkyl, alkyleneoxyalkylaminoalkyl, and the like.

The term “heterocyclyl” as used herein includes molecular fragments or radicals comprising a monovalent chain of carbon atoms and heteroatoms, wherein the heteroatoms are selected from nitrogen, oxygen, and sulfur, a portion of which, including at least one heteroatom, form a ring, such as aziridine, pyrrolidine, oxazolidine, 3-methoxypyrrolidine, 3-methylpiperazine, and the like. Accordingly, as used herein, heterocyclyl includes alkylheterocyclyl, heteroalkylheterocyclyl, heterocyclylalkyl, heterocyclylheteroalkyl, and the like. It is to be understood that the term heterocyclyl as used herein includes fragments and radicals attached at either ring atoms or non-ring atoms, such as tetrahydrofuran-2-yl, piperidin-1-yl, piperidin-4-yl, piperazin-1-yl, morpholin-1-yl, tetrahydrofuran-2-ylmethyl, piperidin-1-ylethyl, piperidin-4-ylmethyl, piperazin-1-ylpropyl, morpholin-1-ylethyl, and the like.

The term “aryl” as used herein includes molecular fragments or radicals comprising an aromatic mono or polycyclic ring of carbon atoms, such as phenyl, naphthyl, and the like.

The term “heteroaryl” as used herein includes molecular fragments or radicals comprising an aromatic mono or polycyclic ring of carbon atoms and at least one heteroatom selected from nitrogen, oxygen, and sulfur, such as pyridinyl, pyrimidinyl, indolyl, benzoxazolyl, and the like.

The term “substituted aryl” or “substituted heteroaryl” as used herein includes molecular fragments or radicals comprising aryl or heteroaryl substituted with one or more substituents, such as alkyl, heteroalkyl, halo, hydroxy, amino, alkyl or dialkylamino, alkoxy, alkylsulfonyl, aminosulfonyl, carboxylate, alkoxycarbonyl, aminocarbonyl, cyano, nitro, and the like. It is to be understood that the alkyl groups in such substituents may be optionally substituted with halo.

The term “iminoalkylidenyl” as used herein includes molecular fragments or radicals comprising a divalent radical containing alkylene as defined herein and a nitrogen atom, where the terminal carbon of the alkylene is double-bonded to the nitrogen atom, such as the formulae —(CH)═N—, —(CH₂)₂(CH)═N—, —CH₂C(Me)═N—, and the like.

The term “amino acid” as used herein includes molecular fragments or radicals comprising an aminoalkylcarboxylate, where the alkyl radical is optionally substituted with alkyl, hydroxy alkyl, sulfhydrylalkyl, aminoalkyl, carboxyalkyl, and the like, including groups corresponding to the naturally occurring amino acids, such as serine, cysteine, methionine, aspartic acid, glutamic acid, and the like.

For example, in one embodiment, amino acid is a divalent radical having the general formula:

—N(R)—(CR′R″)_{q}—C(O)—

where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from asparagine, aspartic acid, cysteine, glutamic acid, lysine, glutamine, arginine, serine, ornitine, threonine, and the like. In one variation, the amino acid may be selected from phenylalanine, tyrosine, and the like, derivatives thereof, and substituted variants thereof.

The terms “arylalkyl” and “heteroarylalkyl” as used herein includes molecular fragments or radicals comprising aryl and heteroaryl, respectively, as defined herein substituted with a linear or branched alkylene group, such as benzyl, phenethyl, α-methylbenzyl, picolinyl, pyrimidinylethyl, and the like.

It is to be understood that the above-described terms can be combined to generate chemically-relevant groups, such as “haloalkoxyalkyl” referring to for example trifluoromethyloxyethyl, 1,2-difluoro-2-chloroeth-1-yloxypropyl, and the like.

The term “amino acid derivative” as used herein refers generally to aminoalkylcarboxylate, where the amino radical or the carboxylate radical are each optionally substituted with alkyl, carboxylalkyl, alkylamino, and the like, or optionally protected; and the intervening divalent alkyl fragment is optionally substituted with alkyl, hydroxy alkyl, sulfhydrylalkyl, aminoalkyl, carboxyalkyl, and the like, including groups corresponding to the side chains found in naturally occurring amino acids, such as are found in serine, cysteine, methionine, aspartic acid, glutamic acid, and the like.

The term “peptide” as used herein includes molecular fragments or radicals comprising a series of amino acids and amino acid analogs and derivatives covalently linked one to the other by amide bonds.

In another embodiment, the bivalent linker comprises a spacer linker and a releasable linker taken together to form 3-thiosuccinimid-1-ylalkyloxymethyloxy, where the methyl is optionally substituted with alkyl or substituted aryl.

In another embodiment, the bivalent linker comprises a spacer linker and a releasable linker taken together to form 3-thiosuccinimid-1-ylalkylcarbonyl, where the carbonyl forms an acylaziridine with the nucleotide, or analog or derivative thereof.

In another embodiment, the bivalent linker comprises a spacer linker and a releasable linker taken together to form 1-alkoxycycloalkylenoxy.

In another embodiment, the bivalent linker comprises a spacer linker and a releasable linker taken together to form alkyleneaminocarbonyl(dicarboxylarylene)carboxylate.

In another embodiment, the bivalent linker comprises a releasable linker, a spacer linker, and a releasable linker taken together to form dithioalkylcarbonylhydrazide, where the hydrazide forms an hydrazone with the nucleotide, or analog or derivative thereof.

In another embodiment, the bivalent linker comprises a spacer linker and a releasable linker taken together to form 3-thiosuccinimid-1-ylalkylcarbonylhydrazide, where the hydrazide forms an hydrazone with the nucleotide, or analog or derivative thereof.

In another embodiment, the bivalent linker comprises a spacer linker and a releasable linker taken together to form 3-thioalkylsulfonylalkyl(disubstituted silyl)oxy, where the disubstituted silyl is substituted with alkyl or optionally substituted aryl.

In another embodiment, the bivalent linker comprises a plurality of spacer linkers selected from the group consisting of the naturally occurring amino acids and stereoisomers thereof.

In another embodiment, the bivalent linker comprises a releasable linker, a spacer linker, and a releasable linker taken together to form 3-dithioalkyloxycarbonyl or 3-dithioalkyloxycarbonyl, where the carbonyl forms a carbonate with the nucleotide, or analog or derivative thereof.

In another embodiment, the bivalent linker comprises a releasable linker, a spacer linker, and a releasable linker taken together to form 2-dithioalkyloxycarbonyl, where the carbonyl forms a carbonate with the nucleotide, or analog or derivative thereof.

In another embodiment, the bivalent linker comprises a releasable linker, a spacer linker, and a releasable linker taken together to form 3-dithioarylalkyloxycarbonyl, where the carbonyl forms a carbonate with the nucleotide, or analog or derivative thereof, and the aryl is optionally substituted.

In another embodiment, the bivalent linker comprises a spacer linker and a releasable linker taken together to form 3-thiosuccinimid-1-ylalkyloxyalkyloxyalkylidene, where the alkylidene forms an hydrazone with the nucleotide, or analog or derivative thereof, each alkyl is independently selected, and the oxyalkyloxy is optionally substituted with alkyl or optionally substituted aryl.

In another embodiment, the bivalent linker comprises a releasable linker, a spacer linker, and a releasable linker taken together to form 3-dithioalkyloxycarbonylhydrazide.

In another embodiment, the bivalent linker comprises a releasable linker, a spacer linker, and a releasable linker taken together to form 2-dithioalkyloxycarbonylhydrazide.

In another embodiment, the bivalent linker comprises a releasable linker, a spacer linker, and a releasable linker taken together to form 3-dithioalkylamino, where the amino forms a vinylogous amide with the nucleotide, or analog or derivative thereof.

In another embodiment, the bivalent linker comprises a releasable linker, a spacer linker, and a releasable linker taken together to form 3-dithioalkylamino, where the amino forms a vinylogous amide with the nucleotide, or analog or derivative thereof, and the alkyl is ethyl.

In another embodiment, the bivalent linker comprises a releasable linker, a spacer linker, and a releasable linker taken together to form 3-dithioalkylaminocarbonyl, where the carbonyl forms a carbamate with the nucleotide, or analog or derivative thereof.

In another embodiment, the bivalent linker comprises a releasable linker, a spacer linker, and a releasable linker taken together to form 3-dithioalkylaminocarbonyl, where the carbonyl forms a carbamate with the nucleotide, or analog or derivative thereof, and the alkyl is ethyl.

In another embodiment, the bivalent linker comprises a releasable linker, a spacer linker, and a releasable linker taken together to form 3-dithioarylalkyloxycarbonyl, where the carbonyl forms a carbamate or a carbamoylaziridine with the nucleotide, or analog or derivative thereof.

In another embodiment, the polyvalent linker includes spacer linkers and releasable linkers connected to form a polyvalent 3-thiosuccinimid-1-ylalkyloxymethyloxy group, illustrated by the following formula

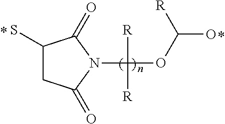

where n is an integer from 1 to 6, the alkyl group is optionally substituted, and the methyl is optionally substituted with an additional alkyl or optionally substituted aryl group, each of which is represented by an independently selected group R. The (*) symbols indicate points of attachment of the polyvalent linker fragment to other parts of the conjugates described herein.

In another embodiment, the polyvalent linker includes spacer linkers and releasable linkers connected to form a polyvalent 3-thiosuccinimid-1-ylalkylcarbonyl group, illustrated by the following formula

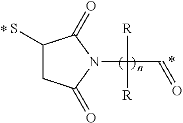

where n is an integer from 1 to 6, and the alkyl group is optionally substituted. The (*) symbols indicate points of attachment of the polyvalent linker fragment to other parts of the conjugates described herein. In another embodiment, the polyvalent linker includes spacer linkers and releasable linkers connected to form a polyvalent 3-thioalkylsulfonylalkyl(disubstituted silyl)oxy group, where the disubstituted silyl is substituted with alkyl and/or optionally substituted aryl groups.

In another embodiment, the polyvalent linker includes spacer linkers and releasable linkers connected to form a polyvalent dithioalkylcarbonylhydrazide group, or a polyvalent 3-thiosuccinimid-1-ylalkylcarbonylhydrazide, illustrated by the following formulae

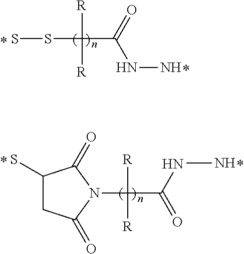

where n is an integer from 1 to 6, the alkyl group is optionally substituted, and the hydrazide forms an hydrazone with (B), (N), or another part of the polyvalent linker (L). The (*) symbols indicate points of attachment of the polyvalent linker fragment to other parts of the conjugates described herein.

In another embodiment, the polyvalent linker includes spacer linkers and releasable linkers connected to form a polyvalent 3-thiosuccinimid-1-ylalkyloxyalkyloxyalkylidene group, illustrated by the following formula

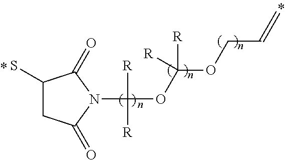

where each n is an independently selected integer from 1 to 6, each alkyl group independently selected and is optionally substituted, such as with alkyl or optionally substituted aryl, and where the alkylidene forms an hydrazone with (B), (N), or another part of the polyvalent linker (L). The (*) symbols indicate points of attachment of the polyvalent linker fragment to other parts of the conjugates described herein.

Additional illustrative linkers are described in WO 2006/012527, the disclosure of which is incorporated herein by reference. Additional linkers are described in the following Table, where the (*) atom is the point of attachment of additional spacer or releasable linkers, the nucleotide, and/or the binding ligand.

Illustrative releasable linkers.

| | |
|---|---|
| 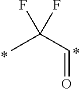 | 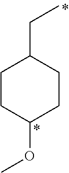 |
| 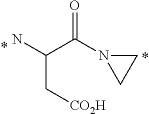 | 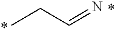 |
| 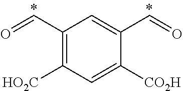 | 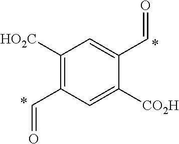 |
| 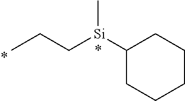 | 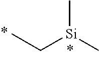 |
| 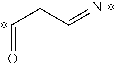 | 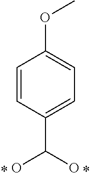 |
| 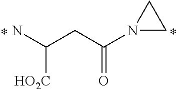 | 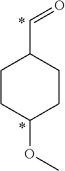 |
| 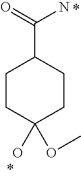 | 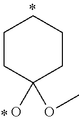 |
| 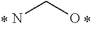 | 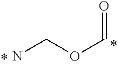 |
| 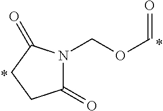 | 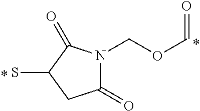 |
| 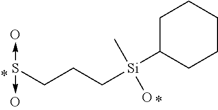 | 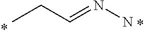 |
| 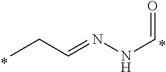 | 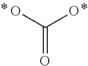 |
| 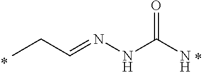 | 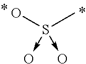 |
| 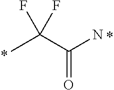 | 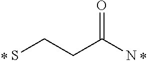 |
| 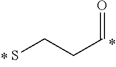 | 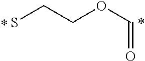 |
| 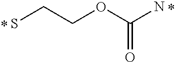 | 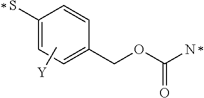 |
| 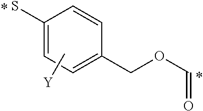 | 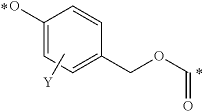 |
| 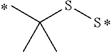 | 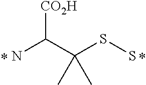 |
| 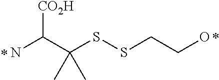 | |

Each of the spacer and releasable linkers described herein is bivalent. In addition, the connections between spacer linkers, releasable linkers, nucleotides N and ligands B may occur at any atom found in the various spacer linkers, releasable linkers, nucleotides N, and ligands B.

The nucleotide can include a nitrogen atom, and the releasable linker can be haloalkylenecarbonyl, optionally substituted with a substituent X², and the releasable linker is bonded to the nucleotide nitrogen to form an amide.

The nucleotide can include an oxygen atom, and the releasable linker can be haloalkylenecarbonyl, optionally substituted with a substituent X², and the releasable linker is bonded to the nucleotide oxygen to form an ester.

The nucleotide can include a double-bonded nitrogen atom, and in this embodiment, the releasable linkers can be alkylenecarbonylamino and 1-(alkylenecarbonylamino)succinimid-3-yl, and the releasable linker can be bonded to the nucleotide nitrogen to form an hydrazone.

The nucleotide can include a sulfur atom, and in this embodiment, the releasable linkers can be alkylenethio and carbonylalkylthio, and the releasable linker can be bonded to the nucleotide sulfur to form a disulfide.

In another embodiment, the binding or targeting ligand capable of binding or targeting PSMA is a phosphoric, phosphonic, or phosphinic acid or derivative thereof. In one aspect, the phosphoric, phosphonic, or phosphinic acid or derivative thereof includes one or more carboxylic acid groups. In another aspect, the phosphoric, phosphonic, or phosphinic acid or derivative thereof includes one or more thiol groups or derivatives thereof. In another aspect, the phosphoric, phosphonic, or phosphinic acid or derivative thereof includes one or more carboxylic acid bioisosteres, such as an optionally substituted tetrazole, and the like.

In another embodiment, the PSMA ligand is a derivative of pentanedioic acid. Illustratively, the pentanedioic acid derivative is a compound of the formula:

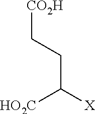

wherein X is RP(O)(OH)CH₂— (see, e.g., U.S. Pat. No. 5,968,915 incorporated herein by reference); RP (O)(OH)N(R¹)— (see, e.g., U.S. Pat. No. 5,863,536 incorporated herein by reference); RP(O)(OH)O— (see, e.g., U.S. Pat. No. 5,795,877 incorporated herein by reference); RN(OH)C(O)Y— or RC(O)NH(OH)Y, wherein Y is —CR₁R₂—, —NR₃— or —O— (see, e.g., U.S. Pat. No. 5,962,521 incorporated herein by reference); RS(O)Y, RSO₂Y, or RS(O)(NH)Y, wherein Y is —CR₁R₂—, —NR₃— or —O— (see, e.g., U.S. Pat. No. 5,902,817 incorporated herein by reference); and RS-alkyl, wherein R is for example hydrogen, alkyl, aryl, or arylalkyl, each of which may be optionally substituted (see, e.g., J. Med. Chem. 46:1989-1996 (2003) incorporated herein by reference).

In each of the foregoing formulae, R, R₁, R₂, and R₃ are each independently selected from hydrogen, C₁-C₉ straight or branched chain alkyl, C₂-C₉ straight or branched chain alkenyl, C₃-C₈ cycloalkyl, C₅-C₇ cycloalkenyl, and aryl. In addition, in each case, each of R, R₁, R₂, and R₃ may be optionally substituted, such as with one or more groups selected from C₃-C₈ cycloalkyl, C₅-C₇ cycloalkenyl, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, amino, aryl. In one aspect, aryl is selected from 1-naphthyl, 2-naphthyl, 2-indolyl, 3-indolyl, 2-furyl, 3-furyl, 2-thienyl, 3-thienyl, 2-pyridyl, 3-pyridyl, 4-pyridyl, benzyl, and phenyl, and in each case aryl may be optionally substituted with one or more, illustratively with one to three, groups selected from halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino. In one variation of each of the above formulae, R is not hydrogen.

Illustrative PSMA ligands described in U.S. Pat. No. 5,968,915 include 2-[[methylhydroxyphosphinyl]methyl]pentanedioic acid; 2-[[ethylhydroxyphosphinyl]methyl]pentanedioic acid; 2-[[propylhydroxyphosphinyl]methyl]pentanedioic acid; 2-[[butylhydroxyphosphinyl]methyl]pentanedioic acid; 2-[[cyclohexylhydroxyphosphinyl]methyl]pentanedioic acid; 2-[[phenylhydroxyphosphinyl]methyl]pentanedioic acid; 2-[[2-(tetrahydrofuranyl)hydroxyphosphinyl]methyl]pentanedioic acid; 2-[[(2-tetrahydropyranyl)hydroxyphosphinyl]methyl]pentanedioic acid; 2-[[((4-pyridyl)methyl)hydroxyphosphinyl]methyl]pentanedioic acid; 2-[[((2-pyridyl)methyl)hydroxyphosphinyl]methyl]pentanedioic acid; 2-[[(phenylmethyl)hydroxyphosphinyl]methyl]pentanedioic acid; 2-[[((2-phenylethyl)methyl)hydroxyphosphinyl]methyl]pentanedioic acid; 2-[[((3-phenylpropyl)methyl)hydroxyphosphinyl]methyl]pentanedioic acid; 2-[[((3-phenylbutyl)methyl)hydroxyphosphinyl]methyl]pentanedioic acid; 2-[[((2-phenylbutyl)methyl)hydroxyphosphinyl]methyl]pentanedioic acid; 2-[[(4-phenylbutyl)hydroxyphosphinyl]methyl]pentanedioic acid; and 2-[[(aminomethyl)hydroxyphosphinyl]methyl]pentanedioic acid.

Illustrative PSMA ligands described in U.S. Pat. No. 5,863,536 include N-[methylhydroxyphosphinyl]glutamic acid; N-[ethylhydroxyphosphinyl]glutamic acid; N-[propylhydroxyphosphinyl]glutamic acid; N-[butylhydroxyphosphinyl]glutamic acid; N-[phenylhydroxyphosphinyl]glutamic acid; N-[(phenylmethyl)hydroxyphosphinyl]glutamic acid; N-[((2-phenylethyl)methyl)hydroxyphosphinyl]glutamic acid; and N-methyl-N-[phenylhydroxyphosphinyl]glutamic acid.

Illustrative PSMA ligands described in U.S. Pat. No. 5,795,877 include 2-[[methylhydroxyphosphinyl]oxy]pentanedioic acid; 2-[[ethylhydroxyphosphinyl]oxy]pentanedioic acid; 2-[[propylhydroxyphosphinyl]oxy]pentanedioic acid; 2-[[butylhydroxyphosphinyl]oxy]pentanedioic acid; 2-[[phenylhydroxyphosphinyl]oxy]pentanedioic acid; 2-[[((4-pyridyl)methyl)hydroxyphosphinyl]oxy]pentanedioic acid; 2-[[((2-pyridyl)methyl)hydroxyphosphinyl]oxy]pentanedioic acid; 2-[[(phenylmethyl)hydroxyphosphinyl]oxy]pentanedioic acid; and 2[[((2-phenylethyl)methyl)hydroxyphosphinyl]oxy]pentanedioic acid.

Illustrative PSMA ligands described in U.S. Pat. No. 5,962,521 include 2-[[(N-hydroxy)carbamoyl]methyl]pentanedioic acid; 2-[[(N-hydroxy-N-methyl)carbamoyl]methyl]pentanedioic acid; 2-[[(N-butyl-N-hydroxy) carbamoyl]methyl]pentanedioic acid; 2-[[(N-benzyl-N-hydroxy)carbamoyl]methyl]pentanedioic acid; 2-[[(N-hydroxy-N-phenyl)carbamoyl]methyl]pentanedioic acid; 2-[[(N-hydroxy-N-2-phenylethyl)carbamoyl]methyl]pentanedioic acid; 2-[[(N-ethyl-N-hydroxy) carbamoyl]methyl]pentanedioic acid; 2-[[(N-hydroxy-N-propyl)carbamoyl]methyl]pentanedioic acid; 2-[[(N-hydroxy-N-3-phenylpropyl)carbamoyl]methyl]pentanedioic acid; 2-[[(N-hydroxy-N-4-pyridyl) carbamoyl]methyl]pentanedioic acid; 2-[[(N-hydroxy)carboxamido]methyl]pentanedioic acid; 2-[[N-hydroxy (methyl)carboxamido]methyl]pentanedioic acid; 2-[[N-hydroxy (benzyl) carboxamido]methyl]pentanedioic acid; 2-[[N-hydroxy(phenyl)carboxamido]methyl]pentanedioic acid; 2-[[N-hydroxy(2-phenylethyl)carboxamido]methyl]pentanedioic acid; 2-[[N-hydroxy(ethyl)carboxamido]methyl]pentanedioic acid; 2-[[N-hydroxy(propyl) carboxamido]methyl]pentanedioic acid; 2-[[N-hydroxy (3-phenylpropyl) carboxamido]methyl]pentanedioic acid; and 2-[[N-hydroxy(4-pyridyl)carboxamido]methyl]pentanedioic acid.

Illustrative PSMA ligands described in U.S. Pat. No. 5,902,817 include 2-[(sulfinyl)methyl]pentanedioic acid; 2-[(methylsulfinyl)methyl]pentanedioic acid; 2-[(ethylsulfinyl)methyl]pentanedioic acid; 2-[(propylsulfinyl)methyl]pentanedioic acid; 2-[(butylsulfinyl)methyl]pentanedioic acid; 2-[(phenylsulfinyl]methyl]pentanedioic acid; 2-[[(2-phenylethyl)sulfinyl]methyl]pentanedioic acid; 2-[[(3-phenylpropyl)sulfinyl]methyl]pentanedioic acid; 2-[[(4-pyridyl)sulfinyl]methyl]pentanedioic acid; 2-[(benzylsulfinyl)methyl]pentanedioic acid; 2-[(sulfonyl)methyl]pentanedioic acid; 2-[(methylsulfonyl)methyl]pentanedioic acid; 2-[(ethylsulfonyl)methyl]pentanedioic acid; 2-[(propylsulfonyl)methyl]pentanedioic acid; 2-[(butylsulfonyl)methyl]pentanedioic acid; 2-[(phenylsulfonyl)methyl]pentanedioic acid; 2-[[(2-phenylethyl)sulfonyl]methyl]pentanedioic acid; 2-[[(3-phenylpropyl)sulfonyl]methyl]pentanedioic acid; 2-[[(4-pyridyl) sulfonyl]methyl]pentanedioic acid; 2-[(benzylsulfonyl)methyl]pentanedioic acid; 2-[(sulfoximinyl)methyl]pentanedioic acid; 2-[(methylsulfoximinyl)methyl]pentanedioic acid; 2-[(ethylsulfoximinyl)methyl]pentanedioic acid; 2-[(propylsulfoximinyl)methyl]pentanedioic acid; 2-[(butylsulfoximinyl)methyl]pentanedioic acid; 2-[(phenylsulfoximinyl]methyl]pentanedioic acid; 2-[[(2-phenylethyl)sulfoximinyl]methyl]pentanedioic acid; 2-[[(3-phenylpropyl) sulfoximinyl]methyl]pentanedioic acid; 2-[[(4-pyridyl)sulfoximinyl]methyl]pentanedioic acid; and 2-[(benzylsulfoximinyl)methyl]pentanedioic acid.

Pentanedioic acid derivatives described herein have been reported to have high binding affinity at PSMA, including but not limited to the following phosphonic and phosphinic acid derivatives

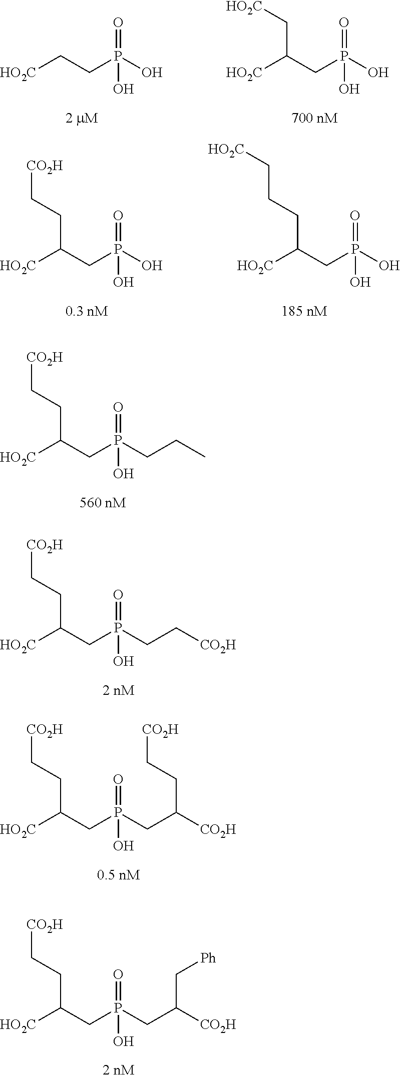

with the dissociation constants (Kᵢ values) shown for the Enzyme-Inhibitor (E-I) complex (see, Current Medicinal Chem. 8:949-0.957 (2001); Silverman, “The Organic Chemistry of Drug Design and Drug Action,” Elsevier Academic Press (2^{nd} Ed. 2003), the disclosures of which are incorporated herein by reference);

In another illustrative embodiment, the pentanedioic acid derivative includes a thiol group, such as compounds of the following formulae:

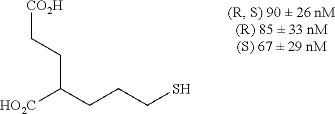

with the inhibition constants (IC₅₀ values) shown for the E-I complex.

In another embodiment, the PSMA ligand is a urea of two amino acids. In one aspect, the amino acids include one or more additional carboxylic acids. In another aspect, the amino acids include one or more additional phosphoric, phosphonic, phosphinic, sulfinic, sulfonic, or boronic acids. In another aspect, the amino acids include one or more thiol groups or derivatives thereof. In another aspect, the amino acids includes one or more carboxylic acid bioisosteres, such as tetrazoles and the like.

In another embodiment, the PSMA ligand is an aminocarbonyl derivative of pentanedioic acid. Illustratively, the aminocarbonylpentanedioic acid derivative is a compound of the formula:

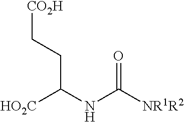

wherein R¹ and R² are each selected from hydrogen, optionally substituted carboxylic acids, such as thiolacetic acids, thiolpropionic acids, and the like; malonic acids, succinic acids, glutamic acids, adipic acids, and the like; and others. Illustrative aminocarbonylpentanedioic acid derivatives are described in J. Med. Chem. 44:298-301(2001) and J. Med. Chem. 47:1729-38 (2004), the disclosures of which are incorporated herein by reference.

In another embodiment, the PSMA ligand is a compound of the formula:

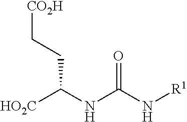

| R¹ | Kᵢ (nM) |
|---|---|
| 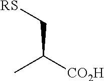 | 6.9 (R = H) 29 (R = tert-Bu) |
| 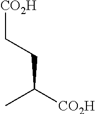 | 8 |
| 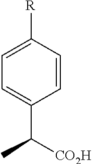 | 2.1 (R = H) 5.9 (R = OH) |
| 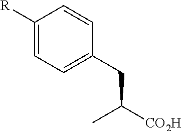 | 12 (R = H) 3.0 (R = OH) |
| 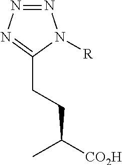 | 0.9 (R = H) 5.3 (R = CH₂CH₂CN) |
| 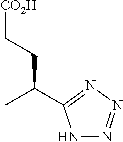 | 335 |

It is appreciated that the urea compounds described herein may also be advantageous in the preparation of the ligands also described herein due to the sub-nanomolar potency, water solubility, and/or long term stability of these compounds. The urea compounds described herein may generally be prepared from commercially available starting materials as described herein.

It is appreciated that in each of the above illustrative pentanedioic acid compounds and urea compounds, there is at least one asymmetric carbon atom. Accordingly, the above illustrative formulae are intended to refer both individually and collectively to all stereoisomers as pure enantiomers, or mixtures of enantiomers and/or diastereomers, including but not limited to racemic mixtures, mixtures that include one epimer at a first asymmetric carbon but allow mixtures at other asymmetric carbons, including racemic mixtures, and the like.

In another illustrative embodiment, the binding agent is a urea of an amino dicarboxylic acid, such as aspartic acid, glutamic acid, and the like, and another amino dicarboxylic acid, or an analog thereof, such as a binding agent of the formulae

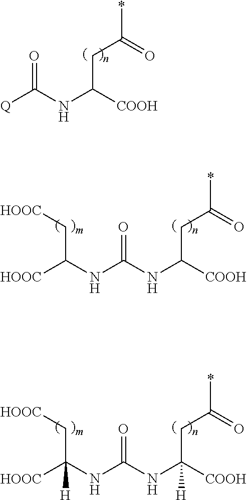

wherein Q is a an amino dicarboxylic acid, such as aspartic acid, glutamic acid, or an analog thereof, n and m are each selected from an integer between 1 and about 6, and (*) represents the point of attachment for the linker L.

In another embodiment, the PSMA ligand includes at least four carboxylic acid groups, or at least three free carboxylic acid groups after the PSMA ligand is conjugated to the agent or linker. It is understood that as described herein, carboxylic acid groups on the PSMA ligand include bioisosteres of carboxylic acids.

Illustratively, the PSMA ligand is a compound of the formulae:

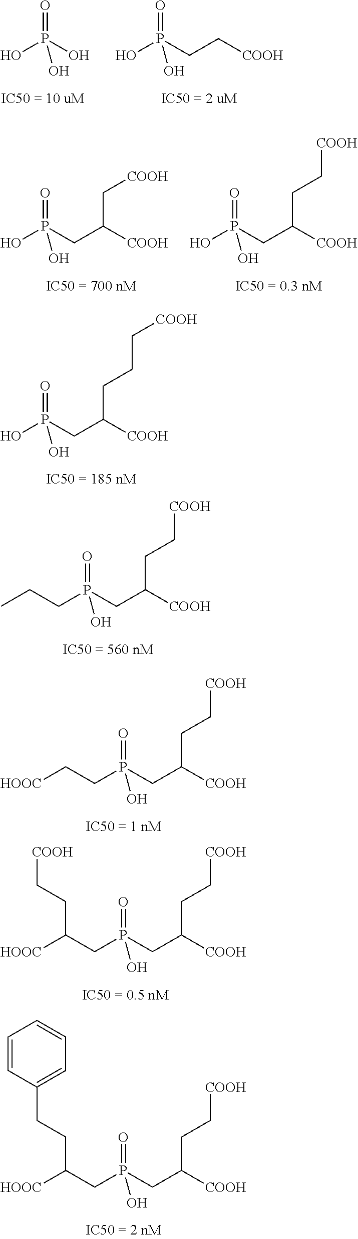

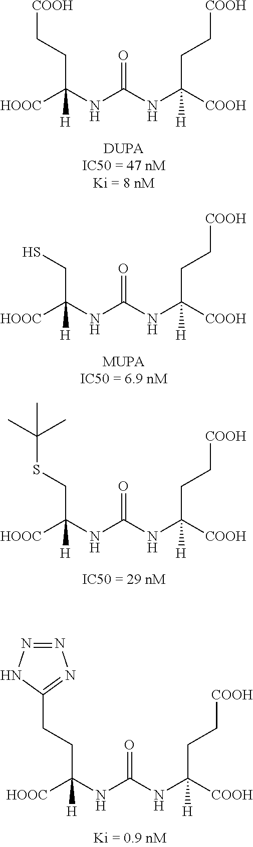

In another embodiment, the PSMA ligand is 2-[3-(1-carboxy-2-mercapto-ethyl)-ureido]-pentanedioic acid (MUPA) or 2-[3-(1,3-dicarboxy-propyl)-ureido]-pentanedioic acid (DUPA)

Other illustrative examples of PSMA ligands include peptide analogs such as quisqualic acid, aspartate glutamate (Asp-Glu), Glu-Glu, Gly-Glu, γ-Glu-Glu, beta-N-acetyl-L-aspartate-L-glutamate (β-NAAG), and the like.

In another illustrative embodiment, the binding agent is a urea of an amino dicarboxylic acid, such as aspartic acid, glutamic acid, and the like, and another amino dicarboxylic acid, or an analog thereof, and the linker is peptide of amino acids, including naturally occurring and non-naturally occurring amino acids. In one embodiment, the linker is a peptide comprising amino acids selected from Glu, Asp, Phe, Cys, beta-amino Ala, and aminoalkylcarboxylic acids, such as Gly, beta Ala, amino valeric acid, amino caproic acid, and the like. In another embodiment, the linker is a peptide consisting of amino acids selected from Glu, Asp, Phe, Cys, beta-amino Ala, and aminoalkylcarboxylic acids, such as Gly, beta Ala, amino valeric acid, amino caproic acid, and the like. In another embodiment, the linker is a peptide comprising at least one Phe. In variations, the linker is a peptide comprising at least two Phe residues, or at least three Phe residues. In another embodiment, the linker is a peptide comprising Glu-Phe or a dipeptide of an aminoalkylcarboxylic acid and Phe. In another embodiment, the linker is a peptide comprising Glu-Phe-Phe or a tripeptide of an aminoalkylcarboxylic acid and two Phe residues. In another embodiment, the linker is a peptide comprising one or more Phe residues, where at least one Phe is about 7 to about 11, or about 7 to about 14 atoms from the binding ligand B. In another embodiment, the linker is a peptide comprising Phe-Phe about 7 to about 11, or about 7 to about 14 atoms from the binding ligand B. It is to be understood that in each of the foregoing embodiments and variations, one or more Phe residues may be replaced with Tyr, or another substituted variation thereof.

In another illustrative embodiment, the binding agent is a urea of an amino dicarboxylic acid, such as aspartic acid, glutamic acid, and the like, and another amino dicarboxylic acid, or an analog thereof, and the linker includes one or more aryl or arylalkyl groups, each of which is optionally substituted, attached to the backbone of the linker. In another embodiment, the linker is a peptide comprising one or more aryl or arylalkyl groups, each of which is optionally substituted, attached to the backbone of the linker about 7 to about 11 atoms from the binding ligand B. In another embodiment, the linker is a peptide comprising two aryl or arylalkyl groups, each of which is optionally substituted, attached to the backbone of the linker, where one aryl or arylalkyl group is about 7 to about 11, or about 7 to about 14 atoms from the binding ligand B, and the other aryl or arylalkyl group is about 10 to about 14, or about 10 to about 17 atoms from the binding ligand B.

As described herein, the conjugates are targeted to cells that express or over-express PSMA, using a PSMA binding ligand. Once delivered, the conjugates bind to PSMA. In some embodiments, the conjugates are internalized in the cell expressing or over-expressing PSMA by endogenous cellular mechanisms, such as endocytosis, for subsequent imaging and/or diagnosis. Once internalized, the conjugates may remain intact or be decomposed, degraded, or otherwise altered to allow the release of the agent forming the conjugate.

In one illustrative embodiment, the nucleotide is an siRNA. In another illustrative variation, the nucleotide is a meRNA. In another illustrative variation, the nucleotide is a segment of double-stranded RNA.

In another aspect, the imaging agent is a fluorescent agent. Fluorescent agents include Oregon Green fluorescent agents, including but not limited to Oregon Green 488, Oregon Green 514, and the like, AlexaFluor fluorescent agents, including but not limited to AlexaFluor 488, AlexaFluor 647, and the like, fluorescein, and related analogs, BODIPY fluorescent agents, including but not limited to BODIPY F1, BODIPY 505, and the like, rhodamine fluorescent agents, including but not limited to tetramethylrhodamine, and the like, DyLight fluorescent agents, including but not limited to DyLight 680, DyLight 800, and the like, CW 800, Texas Red, phycoerythrin, and others. Illustrative fluorescent agent are shown in the following illustrative general structures:

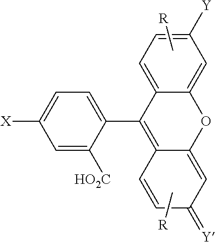

where X is oxygen, nitrogen, or sulfur, and where X is attached to linker L; Y is OR^{a}, NR^{a}₂, or NR^{a}₃⁺; and Y′ is O, NR^{a}, or NR^{a}₂⁺; where each R is independently selected in each instance from H, fluoro, sulfonic acid, sulfonate, and salts thereof, and the like; and R^{a} is hydrogen or alkyl.

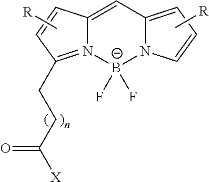

where X is oxygen, nitrogen, or sulfur, and where X is attached to linker L; and each R is independently selected in each instance from H, alkyl, heteroalkyl, and the like; and n is an integer from 0 to about 4.

The binding ligand nucleotide delivery conjugate is preferably administered to the animal parenterally, e.g., intradermally, subcutaneously, intramuscularly, intraperitoneally, intravenously, or intrathecally.

Additionally, more than one type of binding ligand nucleotide delivery conjugate can be used. Illustratively, for example, conjugates with different vitamins, but the same nucleotide can be administered to the animal. In other embodiments, conjugates comprising the same binding ligand linked to different nucleotides, or various binding ligands linked to various nucleotides can be administered to the animal. In another illustrative embodiment, binding ligand nucleotide delivery conjugates with the same or different vitamins, and the same or different nucleotides comprising multiple vitamins and multiple nucleotides as part of the same nucleotide delivery conjugate can be used.

The compounds described herein bind selectively and/or specifically to cells that express or over-express PSMA. In addition, they not only show selectivity between pathogenic cells and normal tissues, they show selectivity among pathogenic cell populations. In addition, the response is specific to PSMA binding as indicated by competition studies conducted with the conjugates described herein where binding is determined with the conjugate alone or in the presence of excess PMPA, a known binding ligand of PSMA. Binding at both the kidney and tumor is blocked in the presence of excess PMPA (see, for example, Method Examples described herein).

In another embodiment, the conjugate has a binding constant K_{d} of about 100 nM or less. In another aspect, the conjugate has a binding constant K_{d} of about 75 nM or less. In another aspect, the conjugate has a binding constant K_{d} of about 50 nM or less. In another aspect, the conjugate has a binding constant K_{d} of about 25 nM or less.

In another embodiment, the conjugates described herein exhibit selectivity for PSMA expressing or PSMA over-expressing cells or tissues relative to normal tissues such as blood, hear, lung, liver, spleen, duodenum, skin, muscle, bladder, and prostate, with at least 3-fold selectivity, or at least 5-fold selectivity. In one variation, the conjugates described herein exhibit selectivity for PSMA expressing or PSMA over-expressing cells or tissues relative to normal tissues with at least 10-fold selectivity. It is appreciated that the selectivity observed for targeting is indicative of the selectivity that may be observed in treating disease states responsive to the selective or specific elimination of cells or cell populations that express or over-express PSMA.

Generally, any manner of forming a conjugate between the bivalent linker (L) and the binding ligand (B), or analog or derivative thereof, between the bivalent linker (L) and the nucleotide, or analog or derivative thereof, including any intervening heteroatoms, can be utilized in accordance with the present invention. Also, any art-recognized method of forming a conjugate between the spacer linker, the releasable linker, and one or more heteroatoms to form the bivalent linker (L) can be used. The conjugate can be formed by direct conjugation of any of these molecules, for example, through hydrogen, ionic, or covalent bonds. Covalent bonding can occur, for example, through the formation of amide, ester, disulfide, or imino bonds between acid, aldehyde, hydroxy, amino, sulfhydryl, or hydrazo groups.

The synthetic methods are chosen depending upon the selection of the optionally included heteroatoms or the heteroatoms that are already present on the spacer linkers, releasable linkers, the nucleotide, and/or the binding ligand. In general, the relevant bond forming reactions are described in Richard C. Larock, “Comprehensive Organic Transformations, a guide to functional group preparations,” VCH Publishers, Inc. New York (1989), and in Theodora E. Greene & Peter G. M. Wuts, “Protective Groups ion Organic Synthesis,” 2d edition, John Wiley & Sons, Inc. New York (1991), the disclosures of which are incorporated herein by reference.

More specifically, disulfide groups can be generally formed by reacting an alkyl or aryl sulfonylthioalkyl derivative, or the corresponding heteroaryldithioalkyl derivative such as a pyridin-2-yldithioalkyl derivative, and the like, with an alkylenethiol derivative. For example, the required alkyl or aryl sulfonylthioalkyl derivative may be prepared according to the method of Ranasinghe and Fuchs, Synth. Commun. 18(3), 227-32 (1988), the disclosure of which is incorporated herein by reference. Other methods of preparing unsymmetrical disulfides are based on reacting an intermediate compound containing a free thiol group with a intermediate containing a thiol reactive group. Illustrative thiol-reactive groups include maleimides, iodoacetamides, activated halogen groups, optionally substituted pyridyl disulfides, thiolsulfonates, vinyl pyridines, vinyl sulfones, acrylates, and aziridino compounds. Other methods of preparing unsymmetrical dialkyl disulfides are based on a transthiolation of unsymmetrical heteroaryl-alkyl disulfides, such as 2-thiopyridinyl, 3-nitro-2-thiopyridinyl, and like disulfides, with alkyl thiol, as described in WO 88/01622, European Patent Application No. 0116208A1, and U.S. Pat. No. 4,691,024, the disclosures of which are incorporated herein by reference. Further, carbonates, thiocarbonates, and carbamates can generally be formed by reacting an hydroxy-substituted compound, a thio-substituted compound, or an amine-substituted compound, respectively, with an activated alkoxycarbonyl derivative having a suitable leaving group.

In various embodiments of the methods, compounds, and compositions described herein, pharmaceutically acceptable salts of the conjugates described herein are described. Pharmaceutically acceptable salts of the conjugates described herein include the acid addition and base salts thereof (e.g., pharmaceutically acceptable salts of a ligand, such as a PSMA binding ligand).

Suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the conjugates described herein are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

In various embodiments of the methods, compounds, and compositions described herein, the PSMA binding ligand nucleotide conjugates may be administered in combination with one or more other drugs (or as any combination thereof).

In one embodiment, the conjugates described herein may be administered as a formulation in association with one or more pharmaceutically acceptable carriers. The carriers can be excipients. The term “carrier” is used herein to describe any ingredient other than a conjugate described herein. The choice of carrier will to a large extent depend on factors such as the particular mode of administration, the effect of the carrier on solubility and stability, and the nature of the dosage form. Pharmaceutical compositions suitable for the delivery of conjugates described herein and methods for their preparation will be readily apparent to those skilled in the art. Such compositions and methods for their preparation may be found, for example, in Remington: The Science & Practice of Pharmacy, 21th Edition (Lippincott Williams & Wilkins, 2005), incorporated herein by reference.

In one illustrative aspect, a pharmaceutically acceptable carrier includes any and all solvents, dispersion media, coatings, antibacterial and antifungal agents, isotonic and absorption delaying agents, and the like, and combinations thereof, that are physiologically compatible. In some embodiments, the carrier is suitable for parenteral administration. Pharmaceutically acceptable carriers include sterile aqueous solutions or dispersions and sterile powders for the extemporaneous preparation of sterile injectable solutions or dispersions. Supplementary active compounds can also be incorporated into compositions of the invention.

In various embodiments, liquid formulations may include suspensions and solutions. Such formulations may comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

In one embodiment, an aqueous suspension may contain the active materials in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or p-hydroxybenzoate; or one or more coloring agents.

In one illustrative embodiment, dispersible powders and granules suitable for preparation of an aqueous suspension by the addition of water provide the active ingredient in admixture with a dispersing or wetting agent, suspending agent and one or more preservatives. Additional excipients, for example, coloring agents, may also be present.

Suitable emulsifying agents may be naturally-occurring gums, for example, gum acacia or gum tragacanth; naturally-occurring phosphatides, for example, soybean lecithin; and esters including partial esters derived from fatty acids and hexitol anhydrides, for example, sorbitan mono-oleate, and condensation products of the said partial esters with ethylene oxide, for example, polyoxyethylene sorbitan monooleate.

In other embodiments, isotonic agents, for example, sugars, polyalcohols such as mannitol, sorbitol, or sodium chloride can be included in the composition. Prolonged absorption of the injectable compositions can be brought about by including in the composition an agent which delays absorption, for example, monostearate salts and gelatin.

In one aspect, a conjugate as described herein may be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. Suitable means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water. In other embodiments, any of the liquid formulations described herein may be adapted for parenteral administration of the conjugates described herein. The preparation of parenteral formulations under sterile conditions, for example, by lyophilization under sterile conditions, may readily be accomplished using standard pharmaceutical techniques well known to those skilled in the art. In one embodiment, the solubility of a conjugate used in the preparation of a parenteral formulation may be increased by the use of appropriate formulation techniques, such as the incorporation of solubility-enhancing agents.

In various embodiments, formulations for parenteral administration may be formulated to be for immediate and/or modified release. In one illustrative aspect, the conjugates of the invention may be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art. In another embodiment, the conjugates described herein or compositions comprising the conjugates may be continuously administered, where appropriate.

In one embodiment, a kit is provided. If a combination of active compounds is to be administered, two or more pharmaceutical compositions may be combined in the form of a kit suitable for sequential administration or co-administration of the compositions. Such a kit comprises two or more separate pharmaceutical compositions, at least one of which contains a conjugate described herein, and means for separately retaining the compositions, such as a container, divided bottle, or divided foil packet. In another embodiment, compositions comprising one or more conjugates described herein, in containers having labels that provide instructions for use of the conjugates are provided.

In one embodiment, sterile injectable solutions can be prepared by incorporating the conjugates in the required amount in an appropriate solvent with one or a combination of ingredients described above, as required, followed by filtered sterilization. Typically, dispersions are prepared by incorporating the conjugates into a sterile vehicle which contains a dispersion medium and any additional ingredients from those described above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The composition can be formulated as a solution, microemulsion, liposome, or other ordered structure. The carrier can be a solvent or dispersion medium containing, for example, water, ethanol, polyol (for example, glycerol, propylene glycol, and liquid polyethylene glycol, and the like), and suitable mixtures thereof. In one embodiment, the proper fluidity can be maintained, for example, by the use of a coating such as lecithin, by the maintenance of the required particle size in the case of dispersion and by the use of surfactants.

Any effective regimen for administering the conjugates can be used. For example, the conjugates can be administered as single doses, or can be divided and administered as a multiple-dose daily regimen. Further, a staggered regimen, for example, one to five days per week can be used as an alternative to daily treatment, and for the purpose of the methods described herein, such intermittent or staggered daily regimen is considered to be equivalent to every day treatment and is contemplated. In one illustrative embodiment the patient is treated with multiple injections of the conjugate to treat tumors or inflammation. In one embodiment, the patient is injected multiple times (preferably about 2 up to about 50 times) with the conjugate, for example, at 12-72 hour intervals or at 48-72 hour intervals. Additional injections of the conjugate can be administered to the patient at an interval of days or months after the initial injections(s) and the additional injections can prevent recurrence of the cancer or inflammation.

Any suitable course of therapy with the conjugate can be used. In one embodiment, individual doses and dosage regimens are selected to provide a total dose administered during a month of about 15 mg. In one illustrative example, the conjugate is administered in a single daily dose administered on M, Tu, W, Th, and F, in weeks 1, 2, and 3 of each 4 week cycle, with no dose administered in week 4. In an alternative example, the conjugate is administered in a single daily dose administered on M, W, and F, of weeks 1, and 3 of each 4 week cycle, with no dose administered in weeks 2 and 4.

The unitary daily dosage of the conjugate can vary significantly depending on the patient condition, the disease state being treated, the molecular weight of the conjugate, its route of administration and tissue distribution, and the possibility of co-usage of other therapeutic treatments such as radiation therapy or additional drugs in combination therapies. The effective amount to be administered to a patient is based on body surface area, mass, and physician assessment of patient condition. Effective doses can range, for example, from about 1 ng/kg to about 1 mg/kg, from about 1 μg/kg to about 500 pg/kg, and from about 1 μg/kg to about 100 μg/kg. These doses are based on an average patient weight of about 70 kg.

The conjugates described herein can be administered in a dose of from about 1.0 ng/kg to about 1000 μg/kg, from about 10 ng/kg to about 1000 μg/kg, from about 50 ng/kg to about 1000 μg/kg, from about 100 ng/kg to about 1000 μg/kg, from about 500 ng/kg to about 1000 μg/kg, from about 1 ng/kg to about 500 μg/kg, from about 1 ng/kg to about 100 μg/kg, from about 1 μg/kg to about 50 μg/kg, from about 1 μg/kg to about 10 μg/kg, from about 5 μg/kg to about 500 μg/kg, from about 10 μg/kg to about 100 μg/kg, from about 20 μg/kg to about 200 μg/kg, from about 10 μg/kg to about 500 μg/kg, or from about 50 μg/kg to about 500 μg/kg. The total dose may be administered in single or divided doses and may, at the physician's discretion, fall outside of the typical range given herein. These dosages are based on an average patient weight of about 70 kg. The physician will readily be able to determine doses for subjects whose weight falls outside this range, such as infants and the elderly.

The conjugates described herein may contain one or more chiral centers, or may otherwise be capable of existing as multiple stereoisomers. Accordingly, it is to be understood that the present invention includes pure stereoisomers as well as mixtures of stereoisomers, such as enantiomers, diastereomers, and enantiomerically or diastereomerically enriched mixtures. The conjugates described herein may be capable of existing as geometric isomers. Accordingly, it is to be understood that the present invention includes pure geometric isomers or mixtures of geometric isomers.

It is appreciated that the conjugates described herein may exist in unsolvated forms as well as solvated forms, including hydrated forms. In general, the solvated forms are equivalent to unsolvated forms and are encompassed within the scope of the present invention. The conjugates described herein may exist in multiple crystalline or amorphous forms. In general, all physical forms are equivalent for the uses contemplated by the present invention and are intended to be within the scope of the present invention.

### Methods and Examples

The compounds described herein may be prepared by conventional organic synthetic methods. In addition, the compounds described herein may be prepared as indicated below. Unless otherwise indicated, all starting materials and reagents are available from commercial supplies. ¹H NMR spectra were obtained using a Bruker 500 MHz cryoprobe, unless otherwise indicated. The DNA sequence 5′-/5AmMC6/CTT ACG CTG AGT ACT TCG ATT-3′ (Oligo₂₁-5′C₆—NH₂) and 5′-/5Cy5/TCG AAG TAC TCA GCG TAA GTT-3′ (Oligo₂₁-5′Cy5) were purchased from IDT, Inc. N-ε-maleimidocaproic acid (ECMA) was obtained from Pierce. Amino acids and triphosgene were purchased from Chem-Impex Int (Chicago, Ill.). N-Hydroxybenzotriazole (HOBt) and O-Benzotriazole-N,N,N′,N′-tetramethyl-uronium-hexafluoro-phosphate (HBTU) were obtained from Peptide Int. (Louisville, Ky.). Palladium-carbon (30%), sodium pyruvate, diisopropylethyl amine (DIPEA), and stannous chloride dihydrate were obtained from Sigma-Aldrich (St. Louis, Mo.). Acetonitrile, triethyl amine (TEA), and trifluoroacetic acid were purchased from Mallinckrodt (Phillipsburg, N.J.) and were used as received. TLC silica gel plates (60 f₂₅₄, 5×10 cm) and silica gel (40-63, 60 Å) were obtained from EMD (Northbrook, Ill.). All other chemicals were purchased from major suppliers. LNCaP cells were obtained from American type culture collection (Rockville, Md.). RPMI 1640 were purchased from Invitrogen (Carlsbad, Calif.). Athymic male nu/nu mice were purchased from NCI Charles River Laboratories (Wilmington, Mass.).

Moisture and oxygen sensitive reactions were carried out under argon atmosphere. Solid phase peptide synthesis was performed under nitrogen atmosphere using standard peptide synthesis apparatus. DCM was distilled under nitrogen from calcium hydride. Flash chromatography purifications were conducted using silica gel; TLC was performed on silica gel TLC plates and visualized with UV light and iodine stain. All the peptides and peptide conjugates were purified using a reverse phase preparative HPLC (Waters, xTerra C₁₈ 10 μm; 19×250 mm) and analyzed using a reverse phase analytical HPLC (Waters, X-Bridge C₁₈ 5 μm; 3.0×50 mm). ¹H spectra were acquired using a Bruker 500 MHz NMR spectrometer equipped with a TXI cryoprobe. Samples were run in 5 mm NMR tubes using CDCl₃ or DMSO-d₆/D₂O solvent. Presaturation was used to reduce the intensity of the residual H₂O peak. All ¹H signals are recorded in ppm with reference to residual CHCl₃ (7.27 ppm) or DMSO (2.50 ppm) and data are reported as (s=singlet, d=doublet, t=triplet, q=quartet, and m=multiplet or unresolved, b=broad, coupling constant in Hz, and integration). The matrix-assisted laser desorption ionization (MALDI) mass spectrometric results were obtained using an Applied Biosystems (Framingham, Mass.) Voyager DE PRO mass spectrometer. The tumor imaging was performed using a Kodak Image Station (In-Vivo FX, Eastman Kodak Company, New Haven, Conn.).

The LNCaP cells were grown as a monolayer using 1640 RPMI medium containing 10% heat-inactivated fetal bovine serum (HIFBS), sodium pyruvate (100 mM) and 1% penicillin streptomycin in a 5% carbon dioxide: 95% air-humidified atmosphere at 37° C.

Mice were maintained on normal rodent chow diet, and housed in polycarbonate shoebox cages with wire top lids in a sterile environment kept on a standard 12 h light-dark cycle for the duration of the study. Maintenance of the animals and the animal studies were performed according to the “NIH animal care and user guidelines” and approved protocol of the Purdue Animal Use and Care Committee (PACUC).

### Example 1

### General Synthesis of PSMA Inhibitor Intermediates for Conjugation

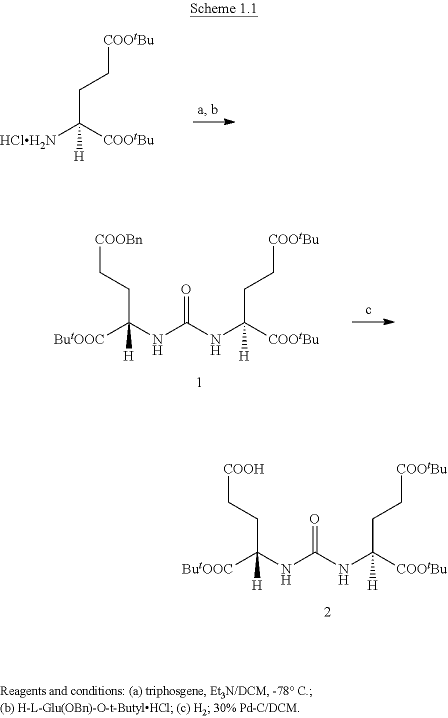

Synthesis Of Urea Compound 2-[3-(3-Benzyloxycarbonyl-1-tert-butoxycarbonyl-propyl)-ureido]-pentanedioic acid di-tert-butyl Ester (1). To a solution of L-glutamate di-tertiary-butylester hydrochloride (1.0 g, 3.39 mmol) and triphosgene (329.8 mg, 1.12 mmol) in dichloromethane (25.0 mL) at −78° C., triethylamine (1.0 mL, 8.19 mmol) was added. After stirring for 2 h at −78° C. under nitrogen, a solution of L-Glu(OBn)-O^{t}Bu (1.2 g, 3.72 mmol) and triethylamine (600 μL, 4.91 mmol) in dichloromethane (5.0 mL) was added. The reaction mixture was allowed to come to room temperature over a period of 1 h and continued to stir at room temperature overnight. The reaction was quenched with 1N HCl, the organic layer was washed with brine and dried over Na₂SO₄. The crude product was purified using a flash chromatography (hexane: EtOAc=1:1) to yield 1(1.76 g, 90.2%) as a colorless oil. R_{f}=0.67 (hexane: EtOAc=1:1); ¹H NMR (CDCl₃) δ 1.43 (s, 9H, CH₃-^{t}Bu); 1.44 (s, 9H, CH₃-^{t}Bu); 1.46 (s, 9H, CH₃-^{t}Bu); 1.85 (m, 1H, Glu-H); 1.87 (m, 1H, Glu-H); 2.06 (m, 1H, Glu-H); 2.07 (m, 1H, Glu-H); 2.30 (m, 2H, Glu-H); 2.44 (m, 2H, Glu-H); 4.34 [s (broad), 1H, αH]; 4.38 [s (broad), 1H, α-H]; 5.10 (s, 2H, CH₂—Ar); 5.22 [s (broad), 2H, Urea-H); 7.34 (m, 5H, Ar—H). HRMS (m/z): (M+H)⁺ calcd. for C₃₀H₄₇N₂O₉, 579.3282. found, 579.3289.

Debenzylation Of Urea Compound 2-[3-(1,3-Bis-tert-butoxycarbonyl-propyl)-ureido]-pentanedioic Acid 1-tert-Butyl Ester (2).

To a solution of 1 (250 mg, 432 mmol) in dichloromethane, 30% Pd/C (50 mg) was added. The reaction mixture was hydrogenated at 1 atm, room temperature for 24 h. Pd/C was filtered through a celite pad and washed with dichloromethane. The crude product was purified using a flash chromatography (hexane: EtOAc=40:60) to yield 2 (169 mg, 80.2%) as a colorless oil. R_{f}=0.58 (hexane: EtOAc=40:60); ¹H NMR (CDCl₃) δ 1.46 (m, 27H, CH₃^{-t}Bu); 1.91 (m, 2H, Glu-H); 2.07 (m, 1H, Glu-H); 2.18 (m, 1H, Glu-H); 2.33 (m, 2H, Glu-H); 2.46 (m, 2H, Glu-H); 4.31 (s (broad), 1H, αH); 4.35 (s (broad), 1H, α-H); 5.05 (t, 2H, Urea-H); HRMS (m/z): (M+H)⁺ calcd. for C₂₃H₄₁N₂O₉, 489.2812. found, 489.2808.

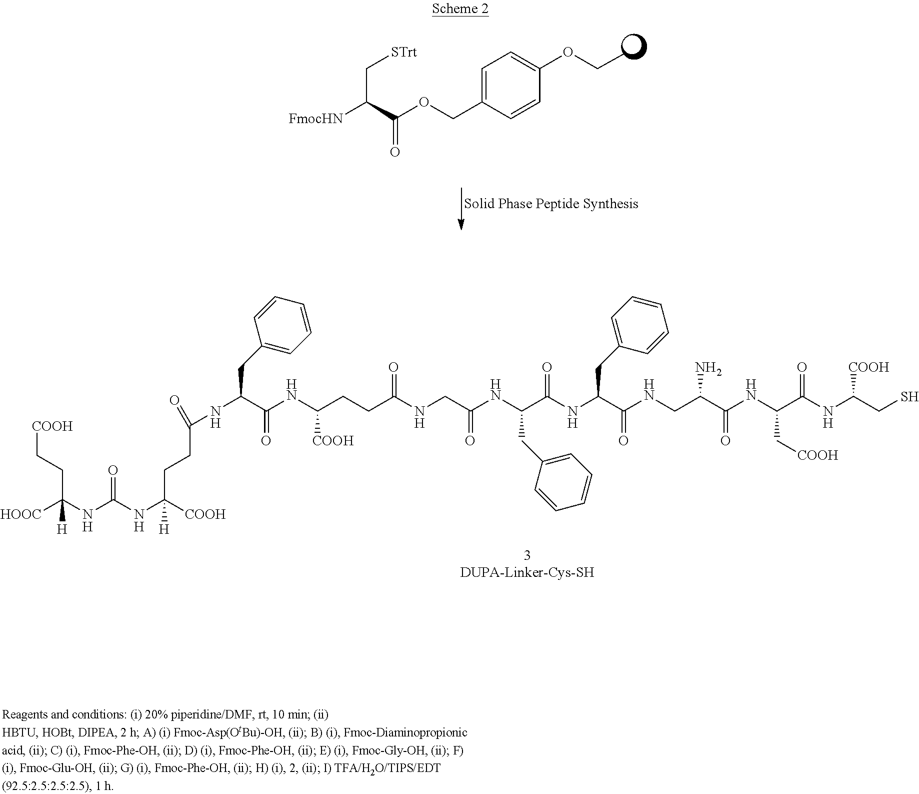

### Example 2

### General Procedure For SPPS of Peptide (3)

Fmoc-Cys(4-methoxytrityl)-Wang resin (100 mg, 0.43 mM) was swelled with DCM (3 mL) followed by DMF (3 mL). A solution of 20% piperidine in DMF (3×3 mL) was added to the resin and nitrogen was bubbled for 5 min. The resin was washed with DMF (3×3 mL) and i-PrOH (3×3 mL). Formation of free amine was assessed by the Kaiser Test. After swelling the resin in DMF, a solution of Fmoc-Asp(OtBu)-OH (2.5 equiv), HBTU (2.5 equiv), HOBt (2.5 equiv), and DIPEA (4.0 equiv) in DMF was added. Argon was bubbled for 2 h, and resin was washed with DMF (3×3 ml) and i-PrOH (3×3 mL). The coupling efficiency was assessed by the Kaiser Test. The above sequence was repeated for 7 more coupling steps. Final compound was cleaved from the resin using trifluoroacitic acid: H₂O: triisopropylsilane: ethanedithiol cocktail and concentrated under vacuum. The concentrated product was precipitated in diethyl ether and dried under vacuum. The crude product was purified using reverse phase preparative HPLC (λ=220 nm; solvent gradient: 1% B to 100% B in 60 min; A=0.1 TFA, B=acetonitrile). Pure fractions were freeze dried to yield 3 as white solid. Analytical HPLC: Rₜ=18.7 min [solvent gradient: 1% B to 100% B 30 min run; HRMS (ESI) (m/z): (M+H)⁺ calcd. for C₅₅H₆₉N₁₁O₂₁S, 1252.4463. found, 1252.4414; UV/Vis: λₘₐₓ=257 nm.

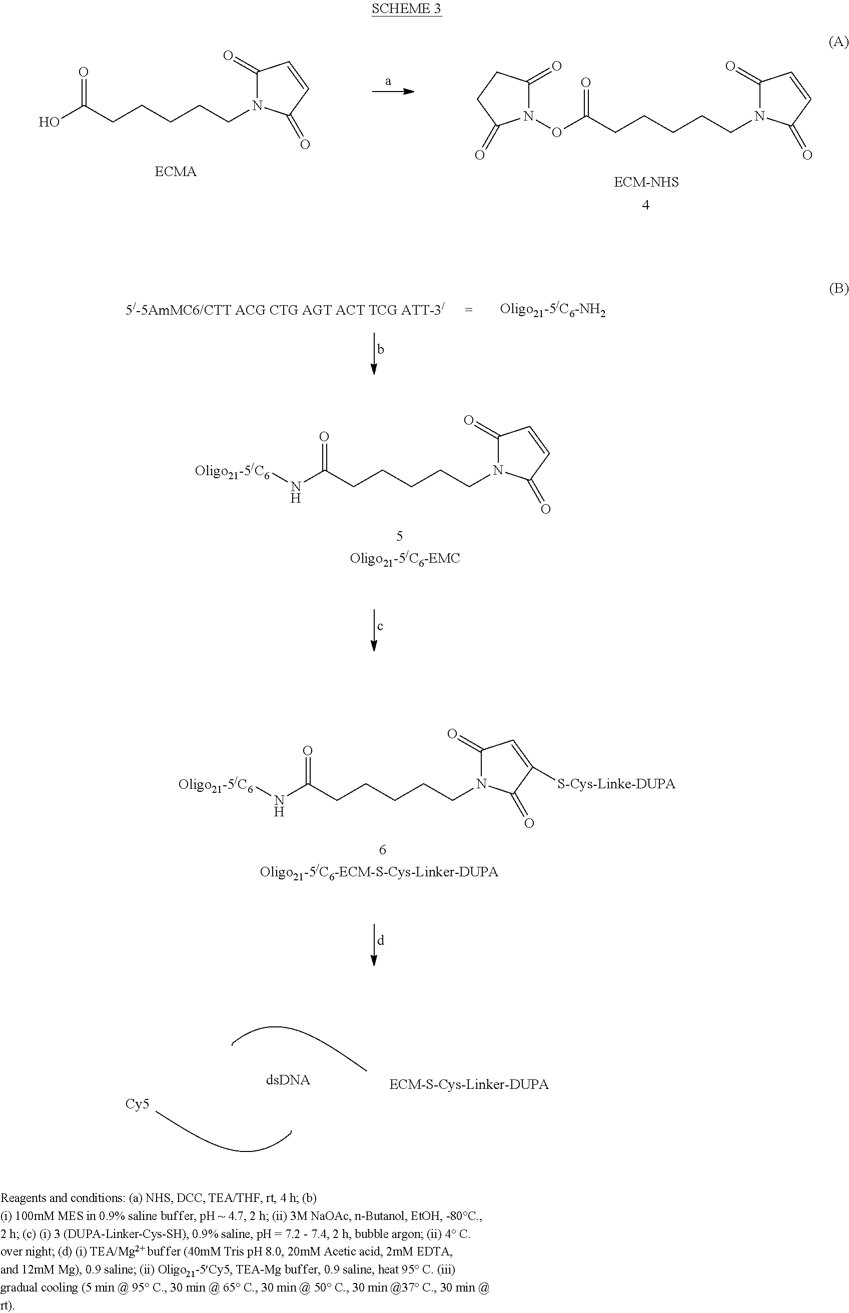

### Example 3

### General Synthesis Of A PSMA Binding Linker-Nucleotide Conjugate

N-ε-maleimidocaproic acid (ECMA; 100 mg, 474 μmol) and N-hydroxysuccinimide (NHS; 81 mg, 710 μmol) were dissolved in tetrahydrofuran (THF). Dicyclohexylcarbodiimide (DCC; 116 mg, 568 μmol) and triethylamine (TEA) were added the reaction mixture, and stirred at room temperature for 4 h. After filtration the urea byproduct, the ECM-NHS was used without further purification. Oligo₂′-5′C₆—NH₂ (2.06 mg, 314 nmol) was dissolved in 100 μL of 2-(N-morpholino)ethanesulfonic acid in 0.9% saline buffer (100 mM, pH 4.7) and ECMA-NHS (0.9 mg, 3.14 μmol) dissolved in 20 μL of DMSO was then added to the reaction mixture. The mixture was agitated for 4 h at room temperature. DUPA-Linke-Cys-SH was dissolved in 0.9% saline (100 μL) and pH of the solution was increased to 7.2 while bubbling argon. Oligo₂′-5′C₆-ECM dissolved in 0.9% saline was added to the reaction mixture and stirred at room temperature for 2 h with bubbling of argon and kept at 4° C. over night. After passing the solution through a gel filtration column (Sephadex G-25), the product was purified by denaturing polyacrylamide gel electrophoresis (PAGE, 20%).

Denaturing Polyacrylamide Gel Electrophoresis [Ko, S; Liu, H.; Chen, Y. & Mao, C. “DNA nanotubes as combinatorial vehicales for cellular delivery” Biomacromolecules, ASAP article (web release)]. Gels contained 20% polyacrylamide (acrylamide/bisacrylamide, 19:1) and 8.3 M urea and were run at 55° C. Tris-borate-EDTA (TBE) was used as the separation buffer and consisted of Tris base (89 mM, pH 8.0), boric acid (89 mM), and EDTA (2 mM). Gels were run on a Hoefer SE 600 electrophoresis unit at 600 V (constant voltage).

Annealing. Oligo₂′-5′C₆-ECM (124 nmol) and Oligo₂′-5′Cy5 (124 nmol) were dissolved in TAE/Mg²⁺ buffer composed of tris(hydroxymethyl) aminomethane (Tris) base (40 mM, pH 8.0), acetic acid (20 mM), ethylenediaminetetraacetate (EDTA; 2 mM), and Mg(OAc)₂ (12.5 mM) in 0.9% saline. Reaction mixture was heat to 95° C. and gradually cooled from 95° C. to 24° C. over 2 h (5 min @ 95° C., 30 min @ 65° C., 30 min @ 50° C., 30 min @ 37° C., 30 min @ rt).

### Example 4A

### General Synthesis of PSMA Imaging Agent Conjugates

Illustrated by synthesis of 14-atom linker compound SK28.

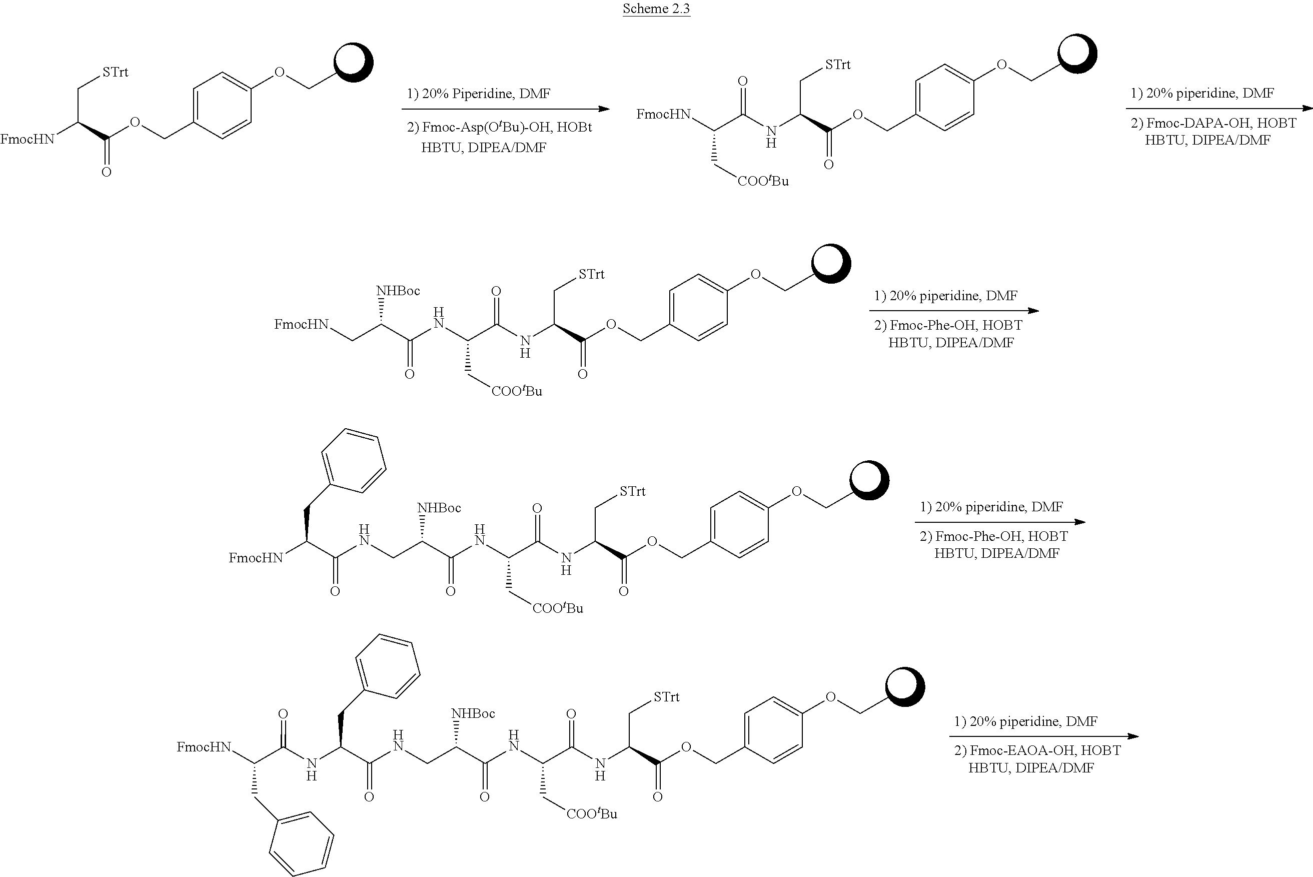

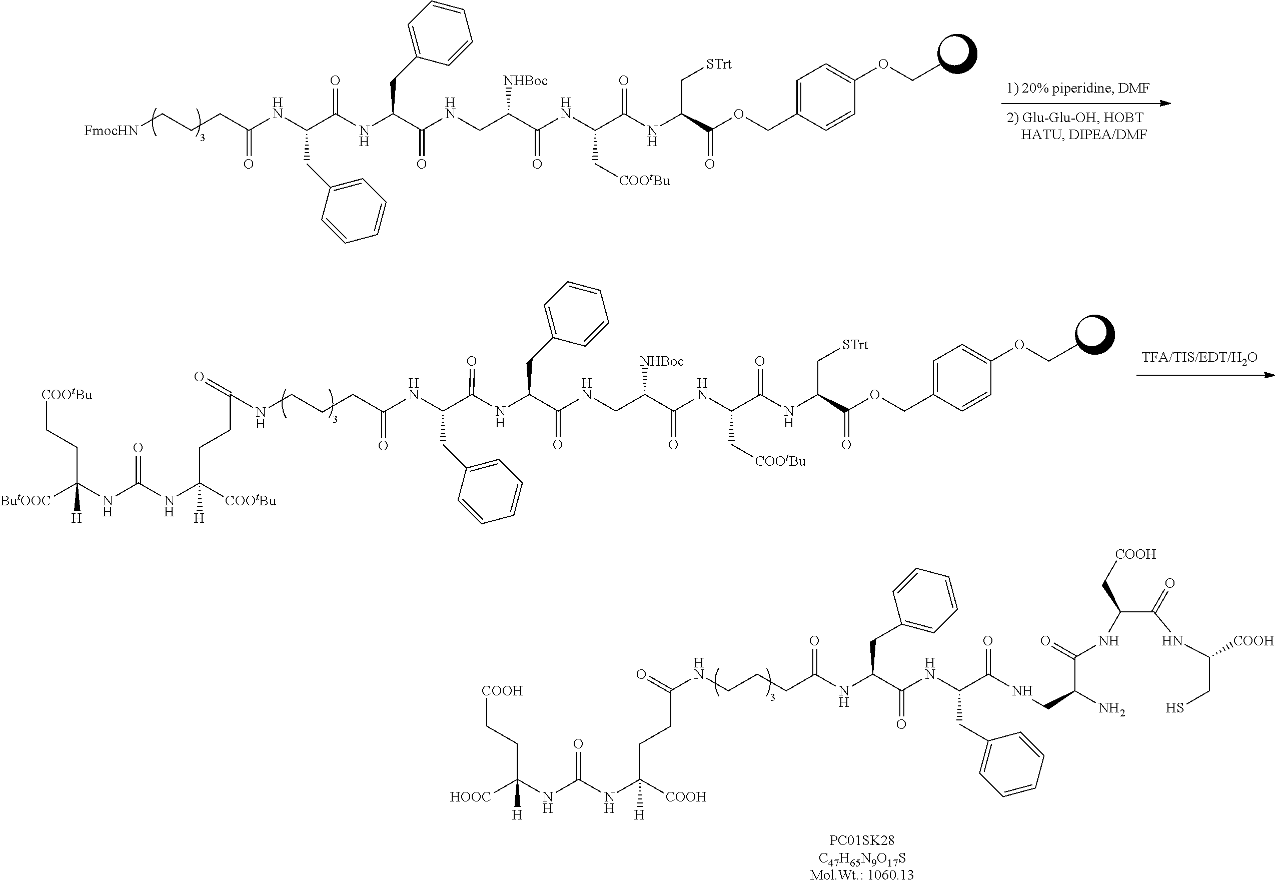

SK28 was synthesized using standard Fluorenylmethyloxycarbonyl (Fmoc) solid phase peptide synthesis (SPPS) starting from Fmoc-Cys(Trt)-Wang resin (Novabiochem; Catalog #04-12-2050). SK28 was purified using reverse phase preparative HPLC (Waters, xTerra C₁₈ 10 μm; 19×250 mm) A=0.1 TFA, B=Acetonitrile (ACN); λ=257 nm; Solvent gradient: 5% B to 80% B in 25 min, 80% B wash 30 min run, (61%). Purified compounds were analyzed using reverse phase analytical HPLC (Waters, X-Bridge C₁₈ 5 μm; 3.0×15 mm); A=0.1 TFA, B=ACN; λ=257 nm, 5% B to 80% B in 10 min, 80% B wash 15 min run. C₄₇H₆₅N₂O₁₇S; MW=1060.13 g/mol; white solid; Rₜ=7.7 min; ¹H NMR (DMSO-d₆/D₂O) δ 0.93 (m, 2H); 1.08 (m, 5H); 1.27 (m, 5H); 1.69 (m, 2H); 1.90 (m, 2H); 1.94 (m, 2H); 2.10 (m, 2H); 2.24 (q, 2H); 2.62 (m, 2H); 2.78 (m, 4H); 2.88 (dd, 1H); 2.96 (t, 2H); 3.01 (dd, 1H); 3.31 (dd, 1H); 3.62 (dd, 1H); 3.80 (q, 1H, αH); 4.07 (m, 1H, αH); 4.37 (m, 1H, αH); 4.42 (m, 2H, αH); 4.66 (m, 1H, αH); 7.18 (m, 10H, Ar—H): LC-MS=1061 (M+H)₊; ESI-MS=1061 (M+H)⁺.

### Examples 4B-4E

The following compounds were synthesized according to the processes described herein using Fmoc SPPS starting from Fmoc-Cys(Trt)-Wang resin (Novabiochem; Catalog #04-12-2050), and purified using reverse phase preparative HPLC (Waters, xTerra C₁₈ 10 μm; 19×250 mm) and analyzed using reverse phase analytical HPLC (Waters, X-Bridge C₁₈ 5 μm; 3.0×15 mm):

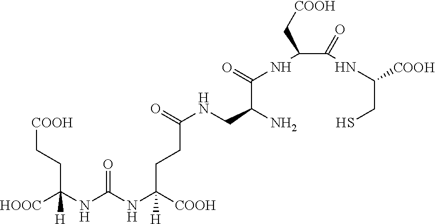

SK60 (0-atom linker): solvent gradient A=0.1 TFA, B=ACN; λ=220 nm; Solvent gradient: 1% B to 50% B in 25 min, 80% B wash 30 min run, (75.3%). C₂₁H₃₂N₆O₁₄S; MW=624.58 g/mol; white solid; Rₜ=6.3 min; ¹H NMR (DMSO-d₆/D₂O) δ 1.70 (m, 2H); 1.92 (m, 2H); 2.17 (m, 2H); 2.23 (m, 2H); 2.57 (m, 1H); 2.77 (m, 4H); 3.45 (dd, 1H); 3.54 (dd, 1H); 3.83 (t, 1H, αH); 4.06 (m, 1H, αH); 4.38 (m, 1H, α-H); 4.63 (m, 1H, α-H); ESI-MS=625 (M+H)⁺

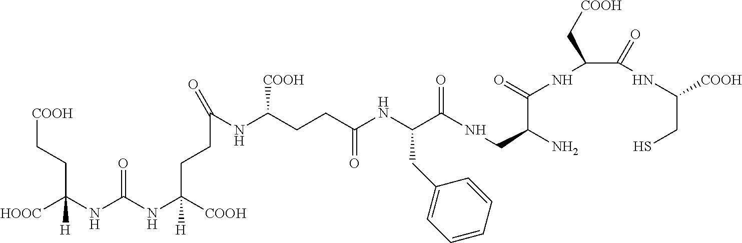

SK62 (7 atom linker): solvent gradient A=0.1 TFA, TFA, B=ACN; λ=220, 257 nm; Solvent gradient: 1% B to 50% B in 25 min, 80% B wash 30 min run, (72%). C₃₅H₄₈N₈O₁₈S; MW=900.86 g/mol; white solid; Rₜ=8.2 min; ¹H NMR (DMOS-d₆/D₂O) δ 1.62 (m, 1H); 1.70 (m, 2H); 1.79 (m, 1H); 1.90 (m, 2H); 2.09 (t, 2H); 2.16 (m, 2H); 2.24 (m, 2H); 2.60 (m, 1H); 2.75 (m, 4H); 2.81 (m, 1H); 2.97 (m, 1H); 3.33 (dd, 1H); 3.60 (dd, 1H); 3.81 (t, 1H, αH); 4.07 (m, 2H, αH); 4.33 [m, 1H, α-H]; 4.39 (t, α-H); 4.65 (m, 1H, α-H); 7.20 (m, 5H, Ar—H); ESI-MS=901 (M+H)⁺.

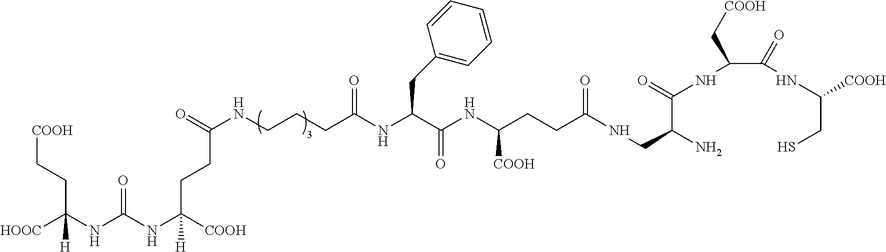

SK38 (16 atom linker): solvent gradient A=10 mM NH₄OAc, B=ACN; λ=257 nm; Solvent gradient: 1% B to 80% B in 25 min, 80% B wash 30 min run, (63%). C₄₃H₆₃N₉O₁₉S, MW=1042.07 g/mol; white solid; Rₜ=min; ¹HNMR (DMSO-d₆/D₂O) δ 0.94 (m, 2H); 1.08 (m, 5H); 1.27 (m, 5H); 1.66 (m, 2H); 1.70 (m, 2H); 1.79 (m, 1H); 1.90 (m, 2H); 2.09 (t, 2H); 2.74 (m, 2H); 2.84 (m, 1H); 2.95 (t, 3H); 3.07 (d, 2H); 3.23 (m, 1H); 3.43 (dd, 1H); 3.52 (dt, 1H); 3.78 (m, 1H, αH); 3.81 (m, 1H, αH); 3.88 (m, 1H, αH); 4.11 (m, 1H, αH); 4.39 [m, 2H, α-H]; 4.65 (m, 1H, α-H); 7.14 (m, 1H, Ar—H); 7.21 (m, 4H, Ar—H): ESI-MS=1043 (M+H)⁺.

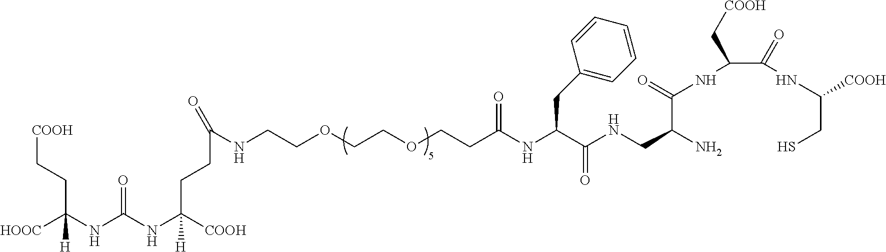

SK57 (24 atom linker): solvent gradient A=0.1 TFA, B=ACN; λ=257 nm; Solvent gradient: 1% B to 50% B in 25 min, 80% B wash 30 min run, (56%). C₄₅H₇₀N₈O₂₂S, MW=1107.14 g/mol; colorless solid; ¹H NMR (DMSO-d₆/D₂O) δ 1.66 (m, 2H); 2.07 (m, 4H); 2.31 (t, 1H); 2.43 (m, 1H); 2.77 (m, 2H); 2.98 (dd, 1H); 3.14 (t, 2H); 3.24 (d, 1H); 3.40 (m, 4H, PEG-H); 3.46 (s, 24H, PEG-H); 3.78 (t, 1H); 3.81 (t, 1H); 4.03 (m, 1H, αH); 4.40 (m, 2H, α-H); 7.16 (m, 1H, Ar—H); 7.22 (m, 4H, Ar—H): ESI-MS=1108 (M+H)⁺.

### Example 4F

The following compound may be synthesized according to the processes described herein.

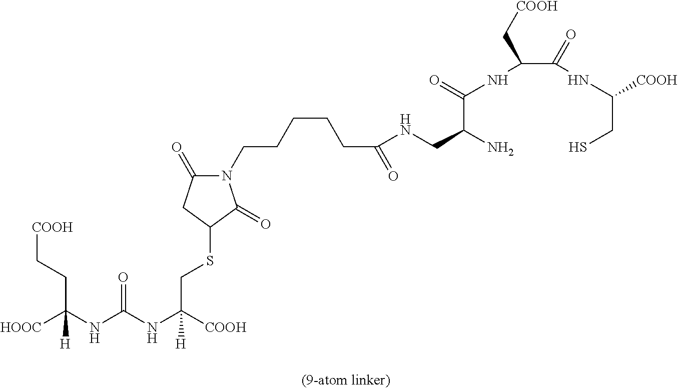

### Example 5A

### General Process for Adding Radionuclide to Chelating Group

Illustrated for radio labeling of SK28 with ^{99m}Tc to prepare SK33.

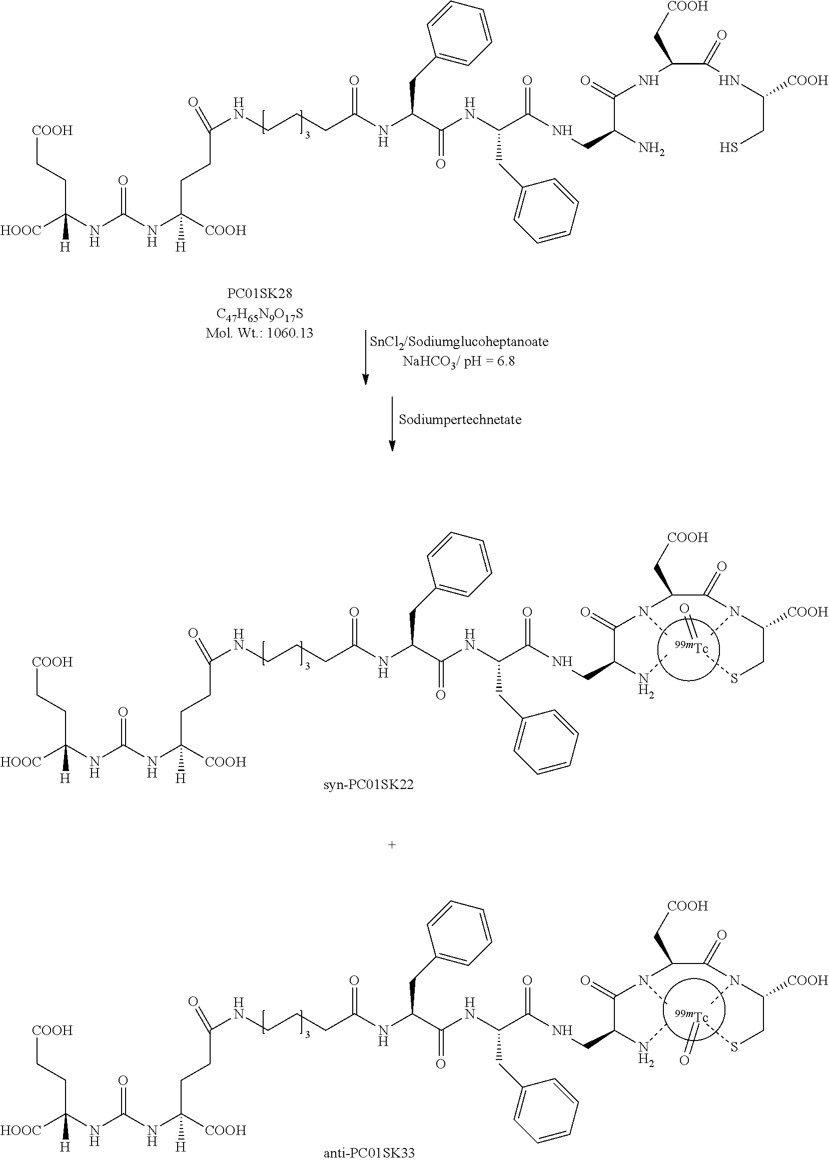

Preparation of SK28 formulation kits. HPLC grade Millipore filtered water (50 mL) was added to a 100 mL bottle and argon was purged for at least 10 min. Sodium α-D-glucoheptonate dihydrate (800 mg) was dissolved in argon purged water (5 mL). Stannous chloride dihydrate (10 mg) was dissolved in 0.02 M HCl (10 mL) while bubbling argon. Stannous chloride (0.8 mL) was added to the sodium glucoheptonate solution under argon. SK28 (1.4 mg) was added to the sodium glucoheptonate/stannous chloride solution under argon. The pH of the reaction mixture was adjusted to 6.8±0.2 using 0.1 N NaOH. Argon purged water (5.2 mL) was added to the reaction mixture to make total volume as 10 mL. 1.0 mL of reaction mixture was dispensed to each vial (10 vials) under argon atmosphere and lyophilized for 36-48 h. The vials were sealed with rubber stoppers and aluminum seals under argon atmosphere to make SK28 formulation kits. The formulation kit vials were stored at −20° C. until they used.

Labeling SK28 with ^{99m}Tc. Radio labeling of SK28 with ^{99m}Tc may be performed according to published procedures. A formulation vial was warmed to room temperature for 10 min and heated in a boiling water bath for 3 min. Then 15 mCi of sodium pertechnetate ^{99m} Tc (1.0 mL) was injected and an equal volume of gas was withdrawn from the vial to normalize the pressure. The vial was heated in the boiling water bath for 15-20 min and then cooled to room temperature before using in the experiment. Radiochemical purity was analyzed by radioactive TLC (>98%), that showed syn and anti isomers of the radio labeled compound (SK33/SK28-^{99m}Tc).

### Examples 5B-5E

The following Examples were prepared according to the processes described herein (both syn and anti isomers were obtained; only the syn isomer is shown):

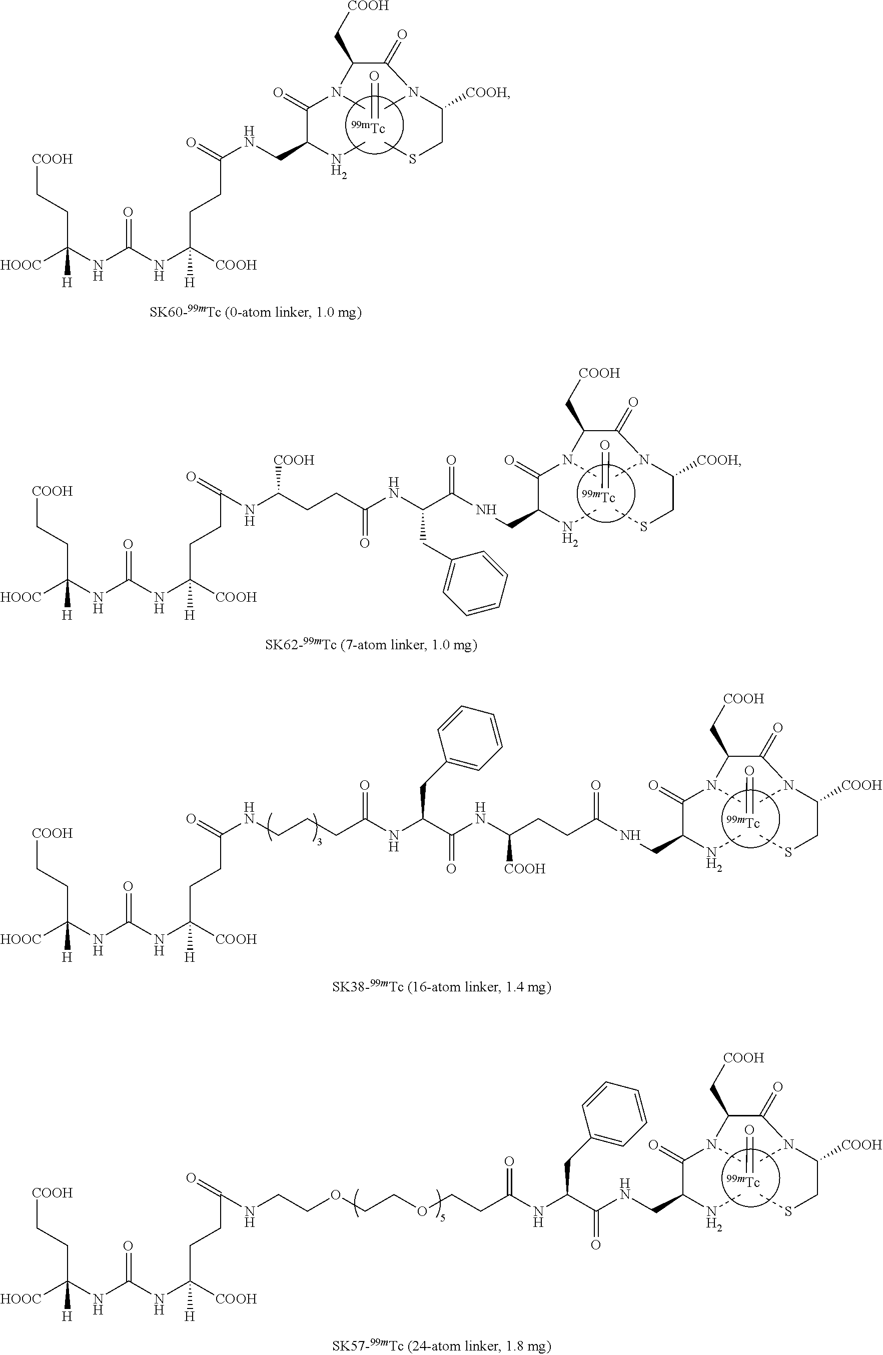

### Example 5F

The following compound may be synthesized according to the processes described herein.

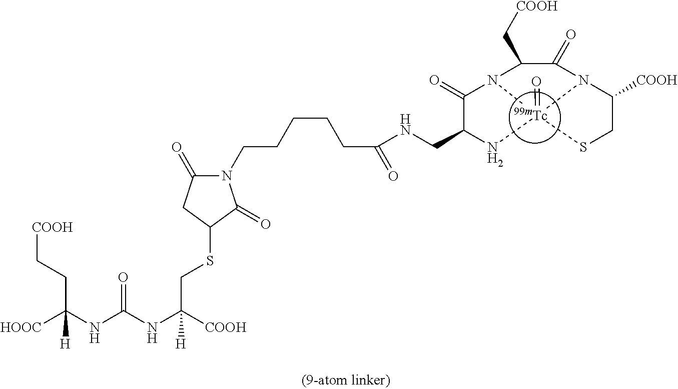

### Example 6

### Confocal Microscopy

LNCaP cells (100,000 cells) were seeded into poly-D-lysine microwell petri dishes (35 mm, 14 mm) and allowed to form monolayers over 24 h. Spent medium was replaced with fresh medium containing DUPA-dsDNA-Cy5 (500 nM) in the presence or absence of 100-fold excess PMPA and cells were incubated for 1 h at 37° C. After rinsing with fresh medium (3×1.0 mL), cells were suspended in fresh medium (1.0 mL). Confocal images were acquired using a Radiance 2100 MP Rainbow (Bio-Rad, Hemel Hempstead, England) on a TE2000 (Nikon, Tokoyo, Japan) inverted microscope using a 60× oil 1.4 NA lens.

### Example 7

### Tumor Models, Imaging, and Bio-Distribution Studies

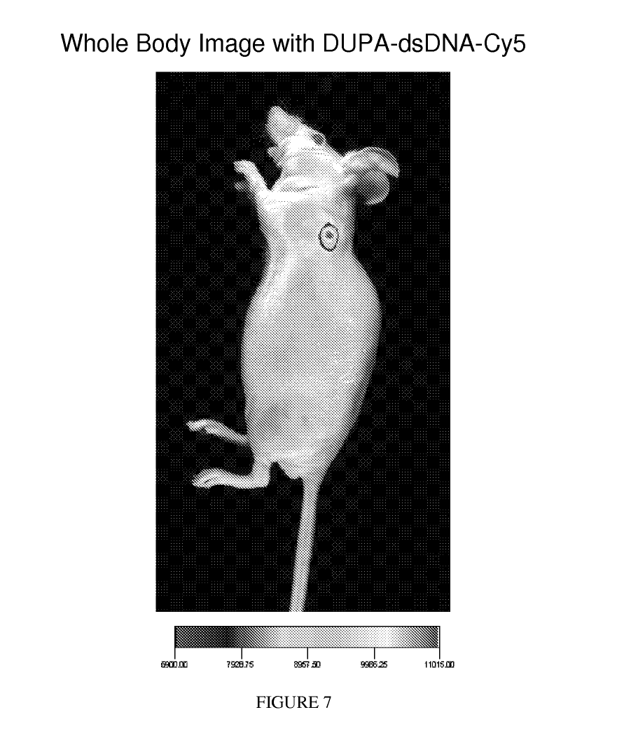

Four- to seven-week-old male nu/nu mice were inoculated subcutaneously with either LNCaP cells [5.0×10⁶/mouse in 1:1 mixture of HC Matrigel and RPMI medium (100 μL)] in the right shoulder. Growth of the tumors was measured in two perpendicular directions every 2 to 3 days using a caliper, and the volumes of the tumors were calculated as 0.5×L×W² (L=measurement of longest axis and W=measurement of axis perpendicular to L in millimeters). Once tumors reached between 200-300 mm³ in volume, animals were treated with DUPA-dsDNA-Cy5 (5 nmol) in saline (200 μL) via lateral tail vein injection. After 3 h, images were acquired by a Kodak Imaging Station (In-Vivo FX, Eastman Kodak Company) in combination with CCD camera and Kodak molecular imaging software (version 4.0). Optical images: illumination source=multilumination, acquisition time=5 sec, f-stop=2.8, focal plane=5, FOV=160, binning=2. White light images: illumination source=white light transillumination, acquisition time=0.05 sec, f-stop=16, focal plane=5, FOV=160 with no binning. See FIG. 7.

### Example 8

### In Vitro Binding Studies Using LNCaP Cells and SK28 (14 Atom Spacer)

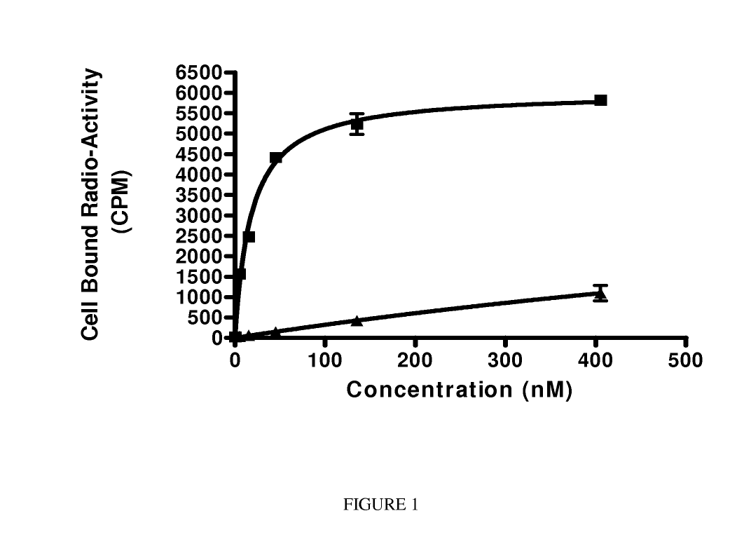

LNCaP cells (a human prostate cancer cell line over-expressing PSMA, purchased from American Type Culture Collection (ATCC)) were seeded in two 24-well (120,000 cells/well) falcon plates and allowed to grow to adherent monolayers for 48 hours in RPMI with glutamine (2 mM) (Gibco RPMI medium 1640, catalog #22400) plus 10% FBS (Fetal Bovine Serum), 1% sodium pyruvate (100 mM) and 1% PS (penicillin streptomycin) in a 5%-CO2 atmosphere at 37° C. Cells of one 24-well plate were incubated with increasing concentrations of SK28-99 mTc from 0 nM-450 nM (triplicates for each concentration) in a 5%-CO2 atmosphere at 37° C. for 1 hour. Cells of the second 24-well plate were incubated with 50 uM PMPA in a 5%-CO2 atmosphere at 37° C. for 30 minutes, then incubated with increasing concentrations of SK28-99 mTc from 0 nM-450 nM (triplicates for each concentration) in a 5%-CO2 atmosphere at 37° C. for 1 hour (competition study). Cells were rinsed three times with 1.0 mL of RPMI. Cells were lysed with tris-buffer, transferred to individual gamma scintigraphy vials, and radioactivity was counted. The plot of cell bound radioactivity verses concentration of radiolabeled compound was used to calculate the Kd value. The competition study was used to determine the binding specificity of the ligand (DUPA) to the PSMA (FIG. 1).

### Example 9

### In Vitro Binding Studies Using LNCaP Cells and SK33 (14 Atom Spacer)

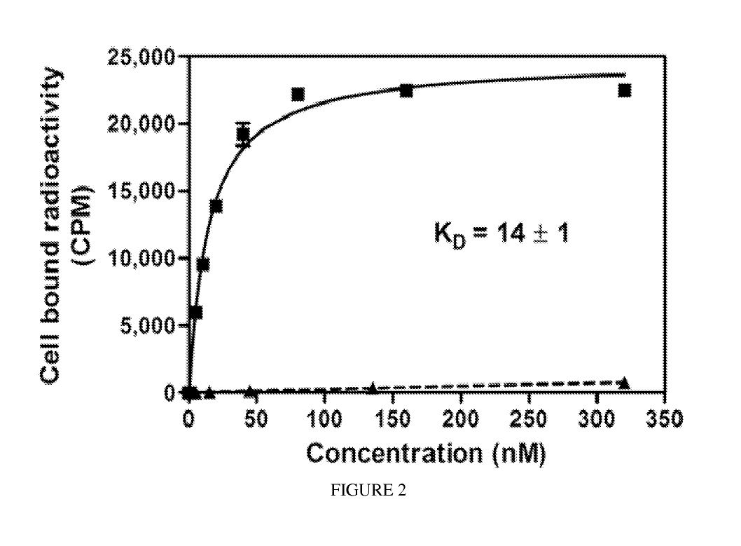

LNCaP cells (150,000 cells/well) were seeded onto 24-well Falcon plates and allowed to form confluent monolayers over 48 h. Spent medium in each well was replaced with fresh medium (0.5 mL) containing increasing concentrations of DUPA-99 mTc in the presence (▴) or absence (▪) of excess PMPA. After incubating for 1 h at 37° C., cells were rinsed with culture medium (2×1.0 mL) and tris buffer (1×1.0 mL) to remove any unbound radioactivity. After suspending cells in tris buffer (0.5 mL), cell bound radioactivity was counted using a γ-counter (Packard, Packard Instrument Company). The dissociation constant (KD) was calculated using a plot of cell bound radioactivity versus the concentration of the radiotracer using nonlinear regression in GraphPad Prism 4. Error bars represent 1 standard deviation (n=3). Experiment was performed three times with similar results. (FIG. 2).

### Example 10

### Quantification of PSMA Molecules on LNCaP Cells

LNCaP cells were seeded in a 24-well falcon plate and allowed to grow to adherent monolayers for 48 hours in RPMI (Gibco RPMI medium 1640, catalog #22400) plus 10% FBS (Fetal Bovine Serum), 1% glutaric and 1% PS (penicillin streptomycin) in a 5%-CO₂ atmosphere at 37° C. Cells were then incubated with increasing concentrations of SK28-99 mTc from 0 nM-450 nM (triplicates for each concentration) in a 5%-CO₂ atmosphere at 4° C. or at 37° C. for 1 hour. Cells were rinsed three times with 1.0 mL of RPMI. Cells were lysed with tris-buffer, transferred to individual gamma scintigraphy vials, and radioactivity was counted. The plot of cell bound radioactivity verses concentration of radiolabeled compound was used to calculate number of PSMA/LNCaP cell. The radioactivity of a 30 nM sample of SK28-99 mTc (20 uL) was counted. At 4° C. (to prevent endocytosis of PSMA), the number of moles in the 30 nM sample=30 nM×20 uL=(30×10-9 mol/L)×(20×10-6 L)=6×10-13 mol. The number of atoms in the 30 nM sample=(6×10-13 mol)×(6.023×1023 atom/mol)=3.6×1011 atom. The radio count of 20 uL of the 30 nM sample=20477 cpm (cpm/atom=3.6×1011/20477=1.76×107). The cell bound radioactivity at the saturation point at 4° C.=12 000 cpm. The number of atoms at the saturation point=(1.76×107 atom)×(12 000 cpm). The number of cells/well=245,000. The number of PSMA/cell at 4° C.=(2.12×1011)/2.45×105=864 396.4˜0.9×106 PSMA/LNCaP cell.

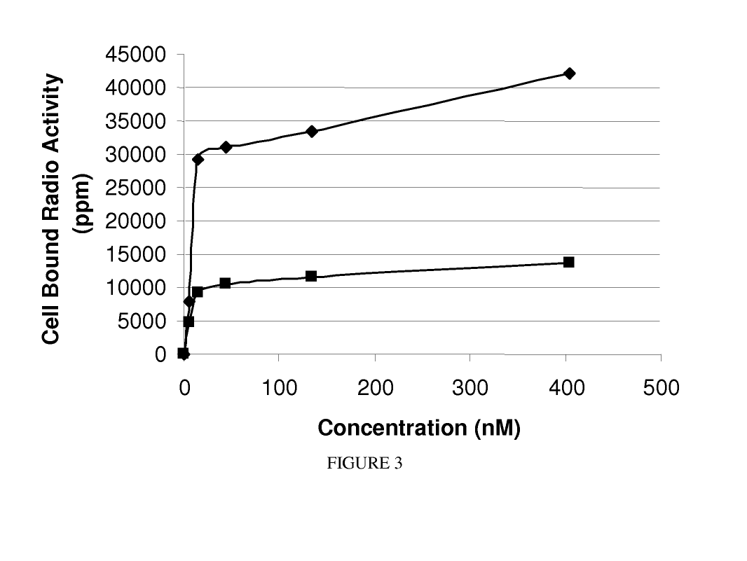

The cell bound radioactivity at the saturation point at 37° C.=33,000 cpm (approximately three fold higher than at 4° C.). This shows that PSMA undergoes endocytosis, unloading the nucleotide and recycling, similar to cell surface receptors. See FIG. 3.

### Example 11

### Spacer-Dependent Binding Studies

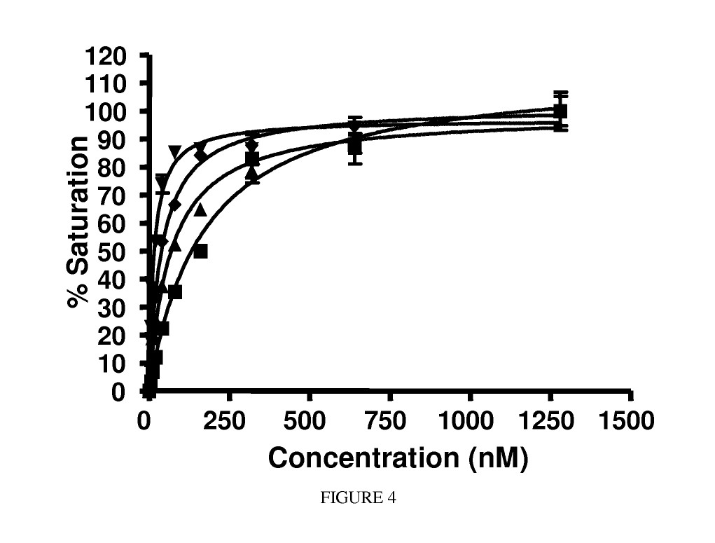

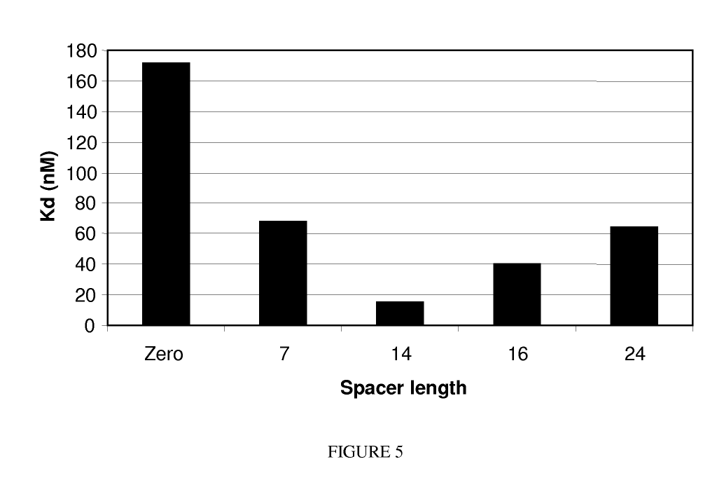

LNCaP cells were seeded in 24-well (120,000 cells/plate) falcon plates (10 plates) and allowed to grow to adherent monolayers for 48 hours in RPMI (Gibco RPMI medium 1640, catalog #22400) plus 10% FBS (Fetal Bovine Serum), 1% sodium pyruvate and 1% PS (penicillin streptomycin) in a 5%-CO₂ atmosphere at 37° C. Cells were then incubated with increasing concentrations of SK60-99 mTc (zero atom spacer), SK62-99 mTc (7 atom spacer), SK28-99 mTc (14 atom spacer), SK38-99 mTc (16 atom spacer) and SK57-99 mTc (24 atom spacer) from 0 nM-1280 nM (triplicates for each concentration) in a 5%-CO₂ atmosphere at 37° C. for 1 hour. Also, in separate plates, cells was incubated with 50 uM PMPA in a 5%-CO₂ atmosphere at 37° C. for 30 minutes and then incubated with increasing concentration of SK60-99 mTc (zero atom spacer), SK62-99 mTc (7 atom spacer), SK28-99 mTc (14 atom spacer), SK38-99 mTc (16 atom spacer) and SK57-99 mTc (24 atom spacer) from 0 nM-1280 nM (triplicates for each concentration) in a 5%-CO2 atmosphere at 37° C. for 1 hour (competition studies; data not shown). Cells were rinsed three times with 1.0 mL of RPMI. Cells were lysed with tris-buffer, transferred to individual gamma scintigraphy vials, and radioactivity was counted. The plot of cell bound radioactivity verses concentration of the radiolabeled compound was used to calculate the Kd value. The plot of % saturation verses concentration of the radiolabeled compound as well as the plot for Kd verses spacer length are shown (FIGS. 4 and 5).

1. A conjugate having the formula
B-L-N
comprising a ligand of PSMA (B), a linker (L), and N, wherein the linker is covalently bound to N and the linker is covalently bound to the ligand, and where the linker comprises a chain of at least seven atoms; and wherein N is selected from the group consisting of single- and double-stranded segments of DNA or RNA, siRNA, microRNA, methylated RNA, iRNA, oligonucleotides, antisense molecules, and ribozymes.

2. (canceled)

3. The conjugate of claim 1 wherein the linker comprises a chain of atoms in the range selected from the group consisting of
from about 7 atoms to about 20 atoms
from about 14 atoms to about 24 atoms, and
from about 14 atoms to about 45 atoms.

4-8. (canceled)

9. The conjugate of claim 1 wherein the linker comprises a chain of atoms in the range from about 10 angstroms to about 45 angstroms in length.

10. (canceled)

11. The conjugate of claim 1 wherein the linker comprises a peptide.

12. The conjugate of claim 1 wherein the linker comprises one or more phenylalanine residues, each of which is independently optionally substituted.

13. (canceled)

14. The conjugate of claim 1 wherein the linker comprises phenylalanyl-phenylalanyl, each phenyl group of which is independently optionally substituted.

15. The conjugate of claim 1 wherein the linker comprises a releasable linker.

16. (canceled)

17. The conjugate of claim 1 wherein the linker comprises a disulfide.

18. The conjugate of claim 1 wherein the linker comprises a releasable linker other than a disulfide.

19. The conjugate of claim 1 wherein the linker comprises a carbonate.

20. The conjugate of claim 1 wherein the linker is non-releasable.

21. The conjugate of claim 1 wherein the ligand comprises a phosphonic acid or a phosphinic acid.

22. The conjugate of claim 1 wherein the ligand is a compound selected from the group consisting of

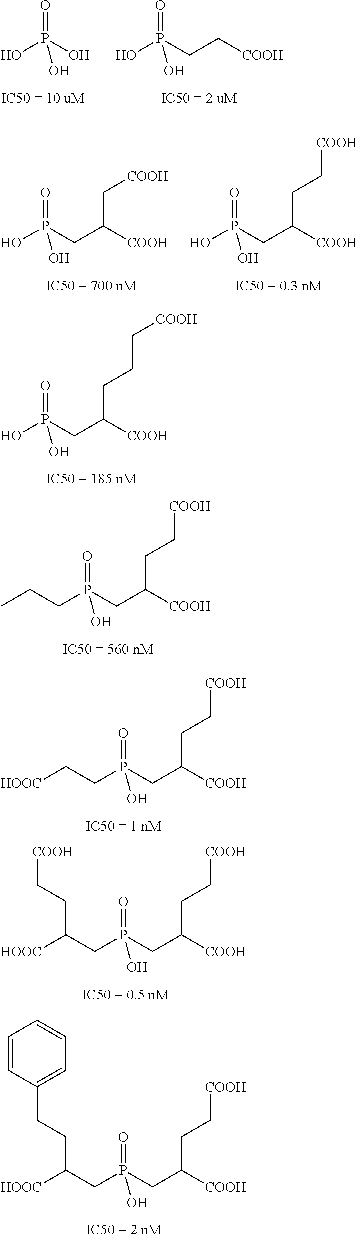

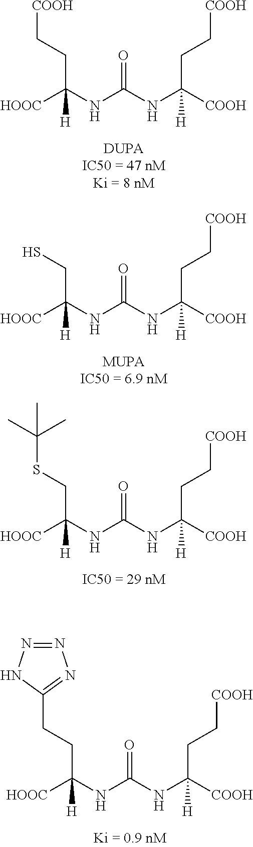

23. The conjugate of claim 1 wherein the ligand is a compound of the formula 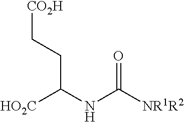
wherein R¹ and R² are each selected from hydrogen, optionally substituted carboxylic acids, such as thiolacetic acids, thiolpropionic acids, and the like; malonic acids, succinic acids, glutamic acids, adipic acids, and the like; and others.

24. The conjugate of claim 1 wherein the ligand is a urea of an amino dicarboxylic acid, and another amino dicarboxylic acid or an analog thereof.

25. The conjugate of claim 1 wherein the ligand is a compound selected from the group consisting of the formulas 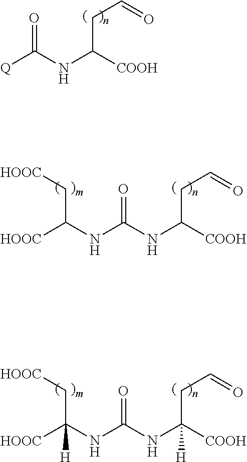
wherein Q is a an amino dicarboxylic acid, or an analog thereof, and n and m are each selected from an integer between 1 and about 6.

26. The conjugate of claim 1 wherein N is selected from the group consisting of siRNA, microRNA, and methylated-RNA.

27. The conjugate of claim 1 wherein N further comprises an imaging agent selected from the group consisting of Oregon Greens, AlexaFluors, fluoresceins, BODIPY fluorescent agents, rhodamines, and DyLight fluorescent agents.

28. A composition a prostate cancer cell targeting effective amount of the conjugate of claim 1, and a component selected from the group consisting of carriers, diluents, and excipients, and combinations thereof.

29. A method for specifically targeting prostate cancer cells in an animal, the method comprising the steps of administering to the animal an effective amount of the conjugate of claim 1, optionally with a component selected from the group consisting of carriers, diluents, and excipients, and combinations thereof; and specifically targeting prostate cancer cells.

30-33. (canceled)
